(19) United States
(12) Patent Application Publication
(10) Pub. No.: US 2021/0002265 A1
(43) Pub. Date: Jan. 7, 2021

(54) PYRAZOLE DERIVATIVES AS INHIBITORS OF THE WNT SIGNALLING PATHWAY

(71) Applicant: Université de Lausanne, Lausanne (CH)

(72) Inventors: Vladimir L. Katanaev, Nyon (CH); Alexey Koval, Le Mont-sur-Lausanne (CH)

(21) Appl. No.: 16/976,882

(22) PCT Filed: Mar. 1, 2019
(86) PCT No.: PCT/EP2019/055117

§ 371 Date: Aug. 31, 2020

(30) Foreign Application Priority Data
Mar. 2, 2018 (EP) 18159649.5

(51) Int. Cl. C07D 405/14; A61P 35/00; C07D 405/10; C07D 401/04

(52) U.S. Cl. CPC C07D 405/14; C07D 401/04; C07D 405/10; A61P 35/00

(57) ABSTRACT

The present invention relates to a novel class of compounds as inhibitors of the Wnt signalling pathway. The best compounds showed potencies in the low micromolar range and high efficacies (>80%) together with good microsomal stability. Furthermore, in vitro characterization of the compounds show promising effects in various anti-cancer assays. Finally, in vivo characterization showed high accumulation in breast tissue.

### TECHNICAL FIELD OF THE INVENTION

The present invention relates to a novel class of compounds as inhibitors of the Wnt signalling pathway. In particular, the present invention relates to the use of this class of compounds in the treatment of cancer, such as triple negative breast cancer. Furthermore, the invention relates to compositions comprising these Wnt pathway inhibitors and their medical use.

### Background of the Invention

Wnt signalling is known to be implicated in various forms of cancer. For example, breast cancer is the most frequently diagnosed and leading cause of death from cancer in women worldwide. It is commonly divided into three major subtypes: the non-mutually exclusive ER+(75%) and HER2+(20%) breast cancer and TNBC (15%). Although TNBC represents the smallest proportion, it is responsible for a disproportionally high amount of breast cancer deaths due to its aggressiveness and rapid growth and recurrence. It is characterized, by the lack of the estrogen, progesterone and human epidermal growth factor receptor 2 (HER2), receptors which are all targets for the currently available drugs. TNBC patients therefore can only rely on surgery, radiotherapy and chemotherapy and novel targeted therapies are urgently needed. Wnt pathway inhibitors have a potential to be used as anti-cancer drugs in general, and particularly against breast cancer, such as TNBC (triple negative breast cancer). This signalling route is one of the essential pathways involved in animal embryonic development, during which it has numerous roles including the regulation of cell proliferation and differentiation. In the healthy adult tissues however, it is largely inactive, with some exceptions such as the renewal of the gastro-intestinal tract, as well as haematopoiesis and regeneration after injury. Aberrant activation of this pathway can lead to diseases of neoplastic nature such as cancer (Nusse, R., Wnt signaling in disease and in development. Cell Res, 2005. 15(1): p. 28-32 and Polakis, P., Drugging Wnt signalling in cancer. EMBO J, 2012. 31(12): p. 2737-46). As the aggressive form of TNBC breast cancer does not respond to the currently available targeted therapy there is an urgent need to develop drugs to combat cancer and in particular this disease (TNBC). Hence, new compounds for the efficient treatment of Wnt pathway dependent cancers such as TNBC breast cancer would be advantageous.

Some reports concerning the Wnt-signalling pathway and also compounds affecting this pathway have been published.

For example, Cases-Selves, M. et al. ChemMedChem, 2017, 12: p. 917-924. investigated the Wnt-signaling inhibition by a series of 1,2,3-thiadiazole-5-carboxamides in a study aiming at deconvolution of the involved mechanism. The authors report that the carboxamides may either inhibit the ATP synthesis through uncoupling of the mitochondrial potential or they may act as ionophores through SERCA2 towards inhibition of the Wnt pathway. The compounds disclosed differ from the present invention.

WO2008/071398 discloses sulfonamides that bind to beta-catenin within the cell nucleus and thereby prevent said beta-catenin from binding to the BCL9 proteins that are associated with the Wnt signaling induced propagation in cancer cells. Special attention is given to sulfonamides of lower molecular weight that demonstrate good cellular permeability as these compounds are expected to perform better than similar and already known Wnt inhibitors acting by the same mechanism. The prior art document does mention treatment of breast cancer, but does not disclose the sulfonamides for treatment of TNBC. The compounds disclosed differ from the present invention.

Ananda, H. et al. Mol Cell Biochem, 2017, 426 p. 149-160. discloses 1-aryl-3,5-bis(het)aryl pyrazole derivatives that they screened in various cancer cells lines to assess their activity on the cell viability. The compounds were found to be cytotoxic against breast adeno-carcinoma cells and leukemic cells. Their investigation revealed that the compounds induce cell death by activation of apoptosis within the cancer cells. The document does not mention the Wnt signaling pathway or how the observed effects may be related thereto. The compounds disclosed differ from the present invention.

Madhavilatha, B. et al. Med Chem Res, 2017, 26, p. 1753-1763 discloses the synthesis of 1,2,3-triazole and isoxazole-linked pyrazole derivatives. The compounds were subsequently evaluated for their anti-proliferative efficacy on four cancer cell lines, including MCF7 breast cancer cells. The document does not mention the Wnt signaling pathway or how the observed effects may be related thereto. The compounds disclosed differ from the present invention.

The present inventors, have screened libraries of small molecules in a transcriptional readout-based screen (TopFlash assay) to identify hit compounds to target the Wnt signalling pathway. Some of the identified molecules were synthesised and further tested for their anti-cancer properties in vitro and in vivo. The inventors surprisingly found, that inhibition of the Wnt signalling pathway by compounds of the invention resulted in e.g. TNBC growth attenuation.

### SUMMARY OF THE INVENTION

Thus, an object of the present invention relates to the identification of novel compounds for inhibition of the Wnt signalling pathway. In particular, it is an object of the present invention to provide novel compounds that inhibit Wnt pathway dependent cancers, such as TNBC breast cancer.

Thus, the first aspect of the invention relates to a compound of formula (I)

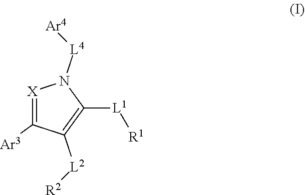

wherein
X is selected from the group consisting of N and CH,
L¹, L², and L⁴ are independently selected from the group consisting of a bond, optionally substituted C₁-C₈ alkylene, optionally substituted C₂-C₈ alkenylene, optionally substituted C₂-C₈ alkynylene, optionally comprising one or more moieties selected from the group consisting of an amide, a thioamide, an ester, an amine, an urea, a carbamate, an aldimine, a ketone and

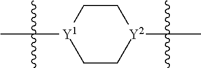

wherein Y¹ and Y² are independently selected from CH and N; or combinations thereof, with the proviso that if L⁴ is a bond, then L² is not a bond,
R¹ and R² are independently selected from the group consisting of H, optionally substituted aryl and optionally substituted heteroaryl,
Ar³ and Ar⁴ are independently selected from the group consisting of optionally substituted aryl and optionally substituted heteroaryl,
   or any pharmaceutically acceptable salt or solvate thereof.

The second aspect of the present invention relates to the compound according the first aspect for use as a medicament.

The third aspect of the present invention relates to the compound according the first aspect for use in the treatment of cancer, particularly triple negative breast cancer.

The fourth aspect of the present invention relates to a method of treating cancer, such as cancers dependent on the Wnt pathway, preferably triple negative breast cancer, said method involving the step of administering a compound according to the first aspect of the invention to a patient in need thereof.

The fifth aspect of the present invention relates to a composition comprising a compound according to the first aspect of the invention and a pharmaceutically acceptable carrier.

The sixth aspect of the present invention relates to a composition comprising the compound according to the first aspect of the invention, an additional pharmaceutically acceptable anti-cancer compound, and a pharmaceutically acceptable carrier.

### BRIEF DESCRIPTION OF THE FIGURES

FIG. 1 shows a depiction of the Wnt signalling pathway.

FIG. 2 shows, Wnt response (% of control) as a function of the concentration of compound 1 (FSA). Wnt-3a whole pathway activation (circle); LiCl activation of downstream elements only (square); Renilla, control for cell well-being (triangle). See also example 3.

FIG. 3 shows ß-catenin stabilization assay comparing effect of compound 1 (50 μM) using either Wnt3a or LiCl. See also example 4.

FIG. 4 shows that compound 1 (50 μM) decreases the stabilization of active ß-catenin in the TNBC cell line HCC 1395 and the total ß-catenin levels in L-cells. See also example 4.

FIG. 5 shows that compound 1 (50 μM) inhibits phosphorylation of DVL in L-cells (left panel) and HCC 1395 cells (TNBC, right panel). See also example 5.

FIG. 6 shows % cells (BT-20, TNBC) in a dose dependent response of compound 1. See also example 6.

FIG. 7 shows % scratch recovery of BT-20, HCC 1806 and MDA-MB 231 TNBC cells with and without the presence of compound 1. See also example 7.

FIG. 8 shows proliferation of HCC 1395, BT-20, HCC 1806, MDA-MB 231 and MDA-MB 468 cells with and without the presence of compound 1. See also example 8.

FIG. 9 shows microsomal stability (CYP [circle] and CYP+UGT [square]) of compound 1. See also example 9.

FIG. 10a-c show in vivo pharmacokinetic profiles of compounds 1 (FSA), 24 (F2-95) and 25 (F2-99). Plasma concentration as function of time is shown. See also example 10.

FIG. 11 shows a synthetic route towards compound 1 (FSA) as described in example 11.

FIG. 12 shows a synthetic route towards compound 24 (F2-99) as described in example 12.

FIG. 13 shows a synthetic route towards compound 25 (F2-95) as described in example 13.

The present invention will now be described in more detail in the following.

### DETAILED DESCRIPTION OF THE INVENTION

### Definitions

Prior to discussing the present invention in further details, the following terms and conventions will first be defined:

In the present context C₁-C₁₀ alkyl is to be understood as univalent groups derived from alkanes (CₙH₂ₙ₊₂) or cycloalkanes (CₙH₂ₙ) by removal of a hydrogen atom from any carbon atom where n is 1-10, i.e. 1-10 carbon atoms are comprised. C₁-C₁₀ alkyls may be linear (—CₙH₂ₙ₊₁), branched (—CₙH₂ₙ₊₁) or cyclic (—CₙH₂ₙ₋₁). The groups derived by removal of a hydrogen atom from a terminal carbon atom of unbranched alkanes form a subclass of normal alkyl (n-alkyl) groups (H(CH₂)ₙ—). Cₓ-C_{y}, such as C₁-C₁₀ generally refers to the total number of carbon atoms also for alkenyls, alkynyls, alkylene, alkenylene and alkynylene. C₂-C₁₀ alkenyls and alkynyls may be linear or branched and C₃-C₁₀ alkenyls may be cyclic. Furthermore, C₂-C₁₀ alkenyls and alkynyls may contain one or more alkene(s) or alkyne(s).

In the present context alkylene is to be understood as an alkanediyl group not necessarily having the free valencies on adjacent carbon atoms, such as propane-1,3-diyl (—CH₂ CH₂CH₂—) or such as propane-1,2-diyl (—CH(CH₃)CH₂—). Alkenylene and alkynylene should be understood in a similar context as an alkenediyl or alkynediyl comprising at least one double bond (alkene) or triple bond (alkyne) respectively.

A first aspect of the invention relates to a compound of formula (I)

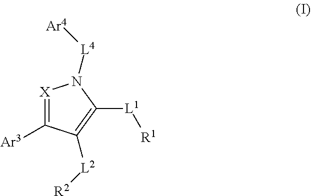

wherein
X is selected from the group consisting of N and CH,
L¹, L², and L⁴ are independently selected from the group consisting of a bond, C₁-C₈ alkylene, C₂-C₈ alkenylene, C₂-C₈ alkynylene, optionally comprising one or more moieties selected from the group consisting of an amide, a thioamide, an ester, an amine, a urea, a carbamate, a aldimine, a ketone and

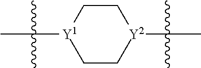

wherein Y¹ and Y² are independently selected from CH and N; or combinations thereof,
R¹ and R² are independently selected from the group consisting of H, optionally substituted aryl and optionally substituted heteroaryl,
Ar³ and Ar⁴ are independently selected from the group consisting of optionally substituted aryl and optionally substituted heteroaryl,
   or any pharmaceutically acceptable salt or solvate thereof.

For L¹, L², and L⁴, the C₁-C₈ alkylene, C₂-C₈ alkenylene, C₂-C₈ alkynylene may independently be optionally substituted.

Preferably, L¹, L², and L⁴ are independently selected from the group consisting of a bond, C₁-C₈ alkylene, C₂-C₈ alkenylene, C₂-C₈ alkynylene, optionally comprising one or more moieties selected from the group consisting of an amide, a thioamide, an ester, an amine, a urea, a carbamate, a aldimine, a ketone and

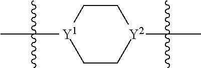

wherein Y¹ and Y² are independently selected from CH and N; or combinations thereof, with the proviso that if L⁴ is a bond, then L² is not a bond.

In the present context “with the proviso that if L⁴ is a bond, then L² is not a bond,” is to be understood in the sense that if L⁴ is a bond (simply connecting the nitrogen of the core heterocycle with Ar⁴) then L² is not a bond, i.e. L² is in these cases selected from the group consisting of C₁-C₈ alkylene, C₂-C₈ alkenylene, C₂-C₈ alkynylene, optionally comprising one or more moieties selected from the group consisting of an amide, a thioamide, an ester, an amine, a urea, a carbamate, a aldimine, a ketone and

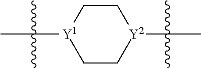

wherein Y¹ and Y² are independently selected from CH and N; or combinations thereof.

In an alternative embodiment L¹, L², and L⁴ are independently selected from the group consisting of a bond, C₁-C₈ alkylene, C₂-C₈ alkenylene, C₂-C₈ alkynylene, optionally comprising one or more moieties selected from the group consisting of an amide, a thioamide, an ester, an amine, a urea, a carbamate, a aldimine, a ketone and

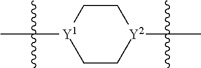

wherein Y¹ and Y² are independently selected from CH and N; or combinations thereof, with the proviso that if L⁴ is a bond, then -L²-R² does not constitute hydrogen (—H).

In the present context, the expression “optionally comprising one or more moieties selected from the group consisting of an amide, a thioamide, an amine, an urea, a carbamate, an aldimine and

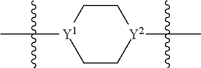

wherein Y¹ and Y² are independently selected from CH and N; or combinations thereof” should be construed as meaning that L¹, L², and L⁴ in addition to the bond, alkylene, alkenylene, alkynylene may comprise one or more of the listed groups within e.g. the alkylene chain, or that in cases where L¹, L², or L⁴ represents a bond, the optional moiety (or moieties) is/are the only moiety or moieties present.

Depending on the nature of the compound of Formula (I) (comprising either an acidic or basic moiety or both) a salt may be formed by addition of a suitable acid or base. The term salt has the usual meaning in the art, as an ionic compound that can be formed by the neutralization reaction of an acid and a base. Salts are composed of related numbers of cations and anions so that the product is electrically neutral. Suitable acids used in salt formation may include but are not limited to hydrogen chloride (HCl), hydrogen bromide (HBr), hydrogen iodide (HI), fumaric acid, maleic acid, citric acid, tartaric acid, salicylic acid, acetic acid, gluconic acid, sulfuric acid (H₂SO₄), methanesulfonic acid (CH₃SO₃H), nitric acid (HNO₃), phosphoric acid (H₃PO₄). Suitable bases used in salt formation may include but are not limited to sodium hydroxide (NaOH), calcium hydroxide (Ca(OH)₂), lithium hydroxide (LiOH), potassium hydroxide (KOH), magnesium hydroxide (Mg(OH)₂), Meglumine, ammonia (NH₃), aluminium hydroxide (Al(OH)₃) and diethanolamine.

In an embodiment of the invention, the pharmaceutically acceptable salt is selected from the group consisting of a chloride salt, bromide salt, iodide salt, fumarate salt, maleate salt, citrate salt, tartrate salt, acetate salt, gluconate salt, sulfate salt, mesylate salt, nitrate salt and phosphate salt.

In another embodiment of the invention the pharmaceutically acceptable salt is selected from the group consisting of a sodium salt, calcium salt, lithium salt, potassium salt, magnesium salt, ammonium salt and an aluminium salt.

Several reasons may prompt the skilled person to make a pharmaceutically acceptable salt of a compound such as improving solubility and/or permeability and/or stability and/or ease of purification. In another embodiment of the invention, a prodrug, such as an ester, of a compound is made. A prodrug has the usual meaning in the art being a medication or compound that, after administration, is metabolized into a pharmacologically active drug. Prodrugs a typical used to improve ADME properties such as poor bioavailability e.g. a drug being poorly absorbed in the gastrointestinal tract.

In an embodiment of the invention, the compound is a crystalline solid. In another embodiment of the invention, the compound is an amorphous solid. Crystalline and amorphous solid has the usual meaning in the art. A crystalline solid thus means any solid material whose constituents are arranged in a highly ordered microscopic structure forming a crystal lattice, i.e. it is the presence of three-dimensional order on the level of atomic dimensions. Crystalline solid may either be single crystals or polycrystals composed of many microscopic crystals also known as crystallites.

A compound may form different crystalline solids (polymorphs) depending on process parameters such as the solvent used during crystallization, whether or not a salt is formed and the type of salt formed. During crystallization or storage a compound may form a solvate or hydrate. A solvate has the usual meaning in the art and is to be understood as a solid with any solvent bound to it. Often the solvate is a hydrate (i.e water bound to the solid). The skilled person is aware that polymorphs as well as solvates/hydrates may have very different properties such as bioavailability.

In an embodiment of the invention said optionally substituted aryl is selected from a 6-, or 10-membered aryl.

In another embodiment of the invention said optionally substituted heteroaryl is selected from a 5-, 6-, 9- or 10-membered heteroaryl, wherein the number of heteroatoms is 1-3, and wherein said heteroatoms are independently selected from the group consisting of N, S, and O.

Aryl and heteroaryl has the usual meaning in the art as groups derived from arenes or heteroarenes by removal of a hydrogen atom from a ring atom. Furthermore, arenes has the usual meaning in the art as being mono- or polycyclic aromatic hydrocarbons. Likewise, heteroarenes are heterocyclic compounds formally derived from arenes by replacement of one or more methine (—C═) and/or vinylene (—CH═CH—) groups by trivalent or divalent heteroatoms, respectively, in such a way as to maintain the continuous n-electron system characteristic of aromatic systems and a number of out-of-plane n-electrons corresponding to the Hückel rule (4 n+2). A heteroatom has the usual meaning in the art as being an atom that is not carbon (C) or hydrogen (H). Typical examples of heteroatoms include but are not limited to nitrogen (N), sulfur (S), oxygen (O), and phosphorus (P).

In embodiment of the invention said optionally substituted aryl or heteroaryl are selected from the group consisting of moieties derived from benzene, naphthalene, pyrrole, furane, thiophene, thiazole, isothiazole, oxazole, isooxazole, pyrazole, imidazole, 1,2,3-oxadiazole, 1,2,4-oxadiazole, 1,2,5-oxadiazole, 1,3,4-oxadiazole, 1,2,3-triazole, 1,2,4-triazole, pyridine, pyridazine, pyrimidine, pyrazine, 1,2,4-triazine, 1,3,5-triazine, 1H-indole, indolizine, 1H-indazole, benzimidazole, 4-azaindole, 5-azaindole, 6-azaindole, 7-azaindole, 7-azaindazole, pyrazolo[1,5-a]pyrimidine, benzofuran, isobenzofuran, benzo[b]thiophene, benzo[c]thiophene, benzo[d]isoxazole, benzo[c]isoxazole, benzo[d]oxazole, benzo[c]isothiazole, benzo[d]thiazole, benzo[c][1,2,5]thiaciazole, 1H-benzotriazole, quinolone, isoquinoline, quinoxaline, phthalazine, quinazoline, cinnoline, 1,8-naphthyridine, pyrido[3,2-d]pyrimidine, pyrido[4,3-d]pyrimidine, pyrido[3,4-b]pyrazine, and pyrido[2,3-b]pyrazine.

In a preferred embodiment of the invention said optionally substituted aryl or heteroaryl are selected from the group consisting of moieties derived from benzene, pyridine, and indole.

In an embodiment of the invention said aryl and heteroaryl may be substituted with one or more substituents, which may be the same or different, and are independently selected from the group consisting of C₁-C₁₀ alkyl, C₂-C₁₀ alkenyl, C₂-C₁₀ alkynyl, phenyl, amino (—NH₂), azido (—N₃), azo C₁-C₁₀ alkyl (—N₂-alkyl), cyanato (—OCN), isocyanato (—NCO), nitroxy (—ONO₂), —CH₂NH(C₁-C₁₀ alkyl), —CH₂N(C₁-C₁₀ alkyl)₂, aminoalkyl (—NH(C₁-C₁₀ alkyl), —N(C₁-C₁₀ alkyl)₂, (—N⁺(C₁-C₁₀ alkyl)₃), 1,3- or 1,4-dioxyl, morpholyl, cyano (—CN), isocyano (—NC), nitroso (—NO), CONH₂, CONH(C₁-C₁₀ alkyl), CON(C₁-C₁₀ alkyl)₂, hydroxyl (—OH), hydroperoxy (—OOH), C₁-C₁₀ peroxy alkyl (—OO-alkyl), C₁-C₁₀ alkyl hydroxyl (-alkyl-OH), C₁-C₁₀ alkoxy (—O-alkyl), carboxylic acid (—COOH), C₁-C₁₀ alkyl esters (—COO-alkyl), oxetanyl, C₁-C₁₀ alkyl acyl (—CO-alkyl), carbamoyloxy (—OC(O)NH₂), —OC(O)NH(C₁-C₁₀ alkyl), —OC(O)N(C₁-C₁₀ alkyl)₂, sulfanyl (—SH), C₁-C₁₀ alkyl thioethers (—S-alkyl), C₁-C₁₀ alkyl thioesters (—C(O)S-alkyl), sulfinic acid (—SO₂H), thiocarboxylic acid (—C(O)SH), sulfonic acid (—SO₃H), C₁-C₁₀ alkyl sulfonate (—SO₃-alkyl), phosphate (—OPO(OH)₂), phosphonic acid (—PO(OH)₂), C₁-C₁₀ alkyl phosphonate (—PO(O-alkyl)₂), phosphinic acid (—P(O)(H)OH), SO₂NH₂, hydroxamic acid (—CONHOH), C₁-C₁₀ alkyl sulfonylureas (—NHCONHSO₂(alkyl)), C₁-C₁₀ acylsulfonamides (—SO₂—NHCO-(alkyl), hydroxyl amine (—NHOH), nitro (—NO₂), imino (—N═CH₂), methyl halide having 1-3 halogen atoms, and halogens; wherein two of said C₁-C₁₀ alkyl and/or said C₁-C₁₀ alkoxy may be linked with a bridge member Z when adjacent, wherein Z is —(CH₂)ₙ—, and n is an integer from 1-6.

Likewise, in an embodiment of the invention L¹, L², and L⁴ may independently be substituted with one or more substituents, which may be the same or different, and are independently selected from the group consisting of C₁-C₁₀ alkyl, C₂-C₁₀ alkenyl, C₂-C₁₀ alkynyl, phenyl, amino (—NH₂), azido (—N₃), azo C₁-C₁₀ alkyl (—N₂-alkyl), cyanato (—OCN), isocyanato (—NCO), nitroxy (—ONO₂), —CH₂NH(C₁-C₁₀ alkyl), —CH₂N(C₁-C₁₀ alkyl)₂, aminoalkyl (—NH(C₁-C₁₀ alkyl), —N(C₁-C₁₀ alkyl)₂, (—N⁺(C₁-C₁₀ alkyl)₃), 1,3- or 1,4-dioxyl, morpholyl, cyano (—CN), isocyano (—NC), nitroso (—NO), CONH₂, CONH(C₁-C₁₀ alkyl), CON(C₁-C₁₀ alkyl)₂, hydroxyl (—OH), hydroperoxy (—OOH), C₁-C₁₀ peroxy alkyl (—OO-alkyl), C₁-C₁₀ alkyl hydroxyl (-alkyl-OH), C₁-C₁₀ alkoxy (—O-alkyl), carboxylic acid (—COOH), C₁-C₁₀ alkyl esters (—COO-alkyl), oxetanyl, C₁-C₁₀ alkyl acyl (—CO-alkyl), carbamoyloxy (—OC(O)NH₂), —OC(O)NH(C₁-C₁₀ alkyl), —OC(O)N(C₁-C₁₀ alkyl)₂, sulfanyl (—SH), C₁-C₁₀ alkyl thioethers (—S-alkyl), C₁-C₁₀ alkyl thioesters (—C(O)S-alkyl), sulfinic acid (—SO₂H), thiocarboxylic acid (—C(O)SH), sulfonic acid (—SO₃H), C₁-C₁₀ alkyl sulfonate (—SO₃-alkyl), phosphate (—OPO(OH)₂), phosphonic acid (—PO(OH)₂), C₁-C₁₀ alkyl phosphonate (—PO(O-alkyl)₂), phosphinic acid (—P(O)(H)OH), SO₂NH₂, hydroxamic acid (—CONHOH), C₁-C₁₀ alkyl sulfonylureas (—NHCONHSO₂(alkyl)), C₁-C₁₀ acylsulfonamides (—SO₂—NHCO-(alkyl), hydroxyl amine (—NHOH), nitro (—NO₂), imino (—N═CH₂), methyl halide having 1-3 halogen atoms, and halogens; wherein two of said C₁-C₁₀ alkyl and/or said C₁-C₁₀ alkoxy may be linked with a bridge member Z when adjacent, wherein Z is —(CH₂)ₙ—, and n is an integer from 1-6.

Optional substituents may generally include homo or hetero polymers constructed from 1-6 monomers of the substituents.

Halogens may include Chlorine (Cl), Bromine (Br), Iodine (I), and Flour (F).

In a preferred embodiment of the invention said aryl and heteroaryl may be substituted with one or more substituents, which may be the same or different, and are independently selected from the group consisting of C₁-C₁₀ alkyl, C₁-C₁₀ alkoxy(—O-alkyl), and halogens; wherein two of said C₁-C₁₀ alkyl and/or said C₁-C₁₀ alkoxy may be linked with a bridge member Z when adjacent, wherein Z is —(CH₂)ₙ—, and n is an integer from 1-6.

In an preferred embodiment of the invention Ar³ and Ar⁴ are independently selected from the group consisting of phenyl,

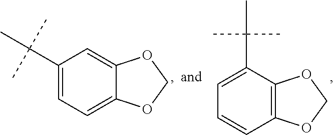

preferably phenyl and

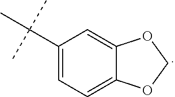

In a preferred embodiment of the invention X is N.

In another preferred embodiment of the invention
R¹ is H or optionally substituted phenyl,
R² is H or optionally substituted 9-membered heteroaryl.

In an embodiment of the invention said C₁-C₈ alkylene, C₂-C₈ alkenylene, and C₂-C₈ alkynylene may be linear or branched, preferably linear. A branched alkylene, alkenylene or alkynylene may comprise any combination possible of primary (R—CH₃), secondary (R—CH₂—R), tertiary (R²CH—R) and/or quaternary (R₃C—R) carbon atoms (R≈H). In another embodiment of the invention, C₃-C₈ alkylene and C₄-C₈ alkenylene may be cyclic. C₃-C₈ alkylene may be cyclic to form a cyclopropane, cyclobutane, cyclopentane, cyclohexane, cycloheptane or cyclooctane. C₄-C₈ alkenylene may be cyclic to form a cyclobutene, cyclopentene, cyclopentadiene, cyclohexene, cyclohexadiene and so forth.

In preferred embodiments L⁴ is not merely a bond, i.e. in one embodiment L⁴ is selected from the group consisting of C₁-C₈ alkylene, C₂-C₈ alkenylene, C₂-C₈ alkynylene, optionally comprising one or more moieties selected from the group consisting of an amide, a thioamide, an ester, an amine, a urea, a carbamate, a aldimine, a ketone and

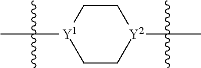

wherein Y¹ and Y² are independently selected from CH and N; or combinations thereof.

In another preferred embodiment L² is a bond and R² is hydrogen.

In an embodiment of the invention L¹, L², and L⁴ are independently selected from the group consisting of a bond, C₁-C₈ alkylene, optionally comprising one or more moieties selected from the group consisting of an amide, a thioamide, an amine, an urea, a carbamate, an aldimine and

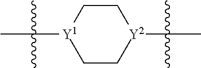

Wherein Y¹ and Y² are independently selected from CH and N; or combinations thereof, with the proviso that if L⁴ is a bond, then L² is not a bond.

In a preferred embodiment of the invention L¹, L², and L⁴ are independently selected from the group consisting of a bond, C₁-C₈ alkylene, a moiety of formula (A)

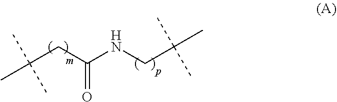

wherein m and p are an integer independently selected from 0-8, with the proviso that m+p is 8 or less, or formula (B)

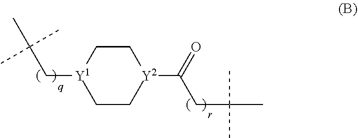

wherein q and r are an integer independently selected from 0-8, with the proviso that q+r is 8 or less, and wherein Y¹ and Y² are independently selected from CH and N, with the proviso that if L⁴ is a bond, then L² is not a bond.

In an more preferred embodiment of the invention
L¹ is a bond,
L² is a bond or a compound of formula (A) wherein m and p are an integer independently selected from 0-4,
L⁴ is a bond or a compound of formula (B) wherein q and r are an integer independently selected from 0-4, and wherein Y¹ is CH and Y² is N,
with the proviso that if L⁴ is a bond, then L² is not a bond.

In yet a more preferred embodiment of the invention
L¹ is a bond,
L² is a bond or a compound of formula (A) wherein m and p are an integer independently selected from 0-4,
L⁴ is a compound of formula (B) wherein q and r are an integer independently selected from 0-4, and wherein Y¹ is CH and Y² is N.

In an even more preferred embodiment of the invention
X is N,
L¹ is a bond,
L² is a bond or a compound of formula (A) wherein m and p are an integer independently selected from 0-4,
L⁴ is a bond or a compound of formula (B) wherein q and r are an integer independently selected from 0-4, and wherein Y¹ is CH and Y² is N,
with the proviso that if L⁴ is a bond, then L² is not a bond.
R¹ is H or optionally substituted phenyl,
R² is H or optionally substituted 9-membered heteroaryl,
Ar³ and Ar⁴ are independently selected from the group consisting of optionally substituted 6-membered aryl and optionally substituted 6-membered heteroaryl,
   wherein the optional substituents, which may be the same or different, are independently selected from the group consisting of C₁-C₁₀ alkyl, C₁-C₁₀ alkoxy(—O-alkyl), and halogens; wherein two of said C₁-C₁₀ alkyl and/or said C₁-C₁₀ alkoxy may be linked with a bridge member Z when adjacent, wherein Z is —(CH₂)ₙ—, and n is an integer from 1-2.

In yet an even more preferred embodiment of the invention
X is N,
L¹ is a bond,
L² is a bond or a compound of formula (A) wherein m and p are an integer independently selected from 0-4,
L⁴ is a compound of formula (B) wherein q and r are an integer independently selected from 0-4, and wherein Y¹ is CH and Y² is N.
R¹ is H or optionally substituted phenyl,
R² is H or optionally substituted 9-membered heteroaryl,
Ar³ and Ar⁴ are independently selected from the group consisting of optionally substituted 6-membered aryl and optionally substituted 6-membered heteroaryl,
   wherein the optional substituents, which may be the same or different, are independently selected from the group consisting of C₁-C₁₀ alkyl, C₁-C₁₀ alkoxy(—O-alkyl), and halogens; wherein two of said C₁-C₁₀ alkyl and/or said C₁-C₁₀ alkoxy may be linked with a bridge member Z when adjacent, wherein Z is —(CH₂)ₙ—, and n is an integer from 1-2.

A more preferred embodiment of the invention relates to compounds of formula (II)

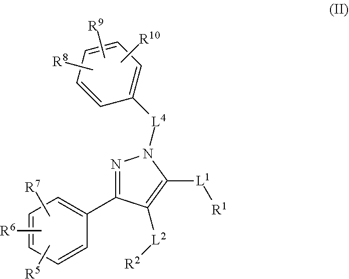

wherein
L¹, L², and L⁴ are as defined in the first aspect,
R¹ and R² are as defined in the first aspect,
R⁵, R⁶, R⁷, R⁸, R⁹ and R¹⁰ may be the same or different and are independently selected from the group consisting of H, C₁-C₁₀ alkyl, C₂-C₁₀ alkenyl, C₂-C₁₀ alkynyl, phenyl, amino (—NH₂), —CH₂NH(C₁-C₁₀ alkyl), —CH₂N(C₁-C₁₀ alkyl)₂, aminoalkyl (—NH(C₁-C₁₀ alkyl) or —N(C₁-C₁₀ alkyl)₂, cyano (—CN), CONH₂, CONH(C₁-C₁₀ alkyl), CON(C₁-C₁₀ alkyl)₂, hydroxyl (—OH), C₁-C₁₀ alkyl hydroxyl (-alkyl-OH), C₁-C₁₀ alkoxy(—O-alkyl), carboxylic acid (—COOH), C₁-C₁₀ alkyl esters (—COO-alkyl), C₁-C₁₀ alkyl acyl (—CO— alkyl), C₁-C₁₀ thioethers (—S-alkyl), sulfonic acid (—SO₃H), C₁-C₁₀ alkyl sulfonate (—SO₃-alkyl), phosphonic acid (—PO(OH)₂), C₁-C₁₀ alkyl phosphonate (—PO(O-alkyl)₂), phosphinic acid (—P(O)(H)OH), SO₂NH₂, hydroxamic acid (—CONHOH), C₁-C₁₀ alkyl sulfonylureas (—NHCONHSO₂(alkyl)), C₁-C₁₀ acylsulfonamides (—SO₂—NHCO-(alkyl), hydroxyl amine (—NHOH), nitro (—NO₂), and halogens; wherein two of said C₁-C₁₀ alkyl and/or said C₁-C₁₀ alkoxy may be linked with a bridge member Z when adjacent, wherein Z is —(CH₂)ₙ—, and n is an integer from 1-6.
   or any pharmaceutically acceptable salt or solvate thereof.

In relation to R⁵, R⁶, R⁷ and R⁸, R⁹ and R¹⁰ it is to be understood that the represent the option of having zero (all R═H), or one to three substituents on the phenyl group to which they are attached. For non-hydrogen substituents they may be in the ortho, para, or meta positions or combinations thereof. Also, as defined for these R groups, two individual R groups may be bridged to form a bicyclic system, particularly when the R groups are alkyl or alkoxy and when they are positioned on adjacent carbons on the phenyl ring. Compounds (IV) and (V) as described infra, are examples of this type of bicyclic system.

In an embodiment of the invention
R⁵, R⁶, R⁷, R⁸, R⁹ and R¹⁰ may be the same or different and are independently selected from the group consisting of H, C₁-C₁₀ alkyl, C₁-C₁₀ alkoxy(—O-alkyl), and halogens; wherein two of said C₁-C₁₀ alkyl and/or said C₁-C₁₀ alkoxy may be linked with a bridge member Z when adjacent, wherein Z is —(CH₂)ₙ—, and n is an integer from 1-6.

In an preferred embodiment of the invention

R⁵, R⁶, R⁷, R⁸ are H, and

R⁹ and R¹⁰ are C₁-C₁₀ alkoxy(—O-alkyl); wherein said C₁-C₁₀ alkoxy may be linked with a bridge member Z when adjacent, wherein Z is —(CH₂)ₙ—, and n is 1.

In preferred embodiments L⁴ is not merely a bond, i.e. in one embodiment L⁴ is selected from the group consisting of C₁-C₈ alkylene, C₂-C₈ alkenylene, C₂-C₈ alkynylene, optionally comprising one or more moieties selected from the group consisting of an amide, a thioamide, an ester, an amine, a urea, a carbamate, a aldimine, a ketone and

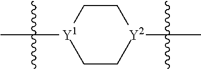

where Y¹ and Y² are independently selected from CH and N; or combinations thereof.

Another preferred embodiment of the invention relates to compounds of formula (III)

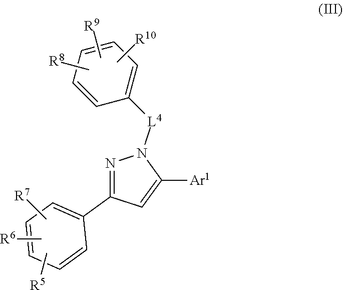

wherein
L⁴ is selected from the group consisting of C₁-C₈ alkylene, C₂-C₈ alkenylene, C₂-C₈ alkynylene, optionally comprising one or more moieties selected from the group consisting of an amide, a thioamide, an ester, an amine, a urea, a carbamate, a aldimine, a ketone and

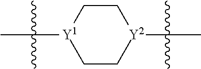

wherein Y¹ and Y² are independently selected from CH and N; or combinations thereof,
R⁵, R⁶, R⁷, R⁸, R⁹ and R¹⁰ are as defined in any of the preceding embodiments,
Ar¹ is selected from the group consisting of optionally substituted phenyl and optionally substituted 5- or 6-membered heteroaryl,
   or any pharmaceutically acceptable salt or solvate thereof.

In an embodiment of the invention Ar¹ is optionally substituted and is selected from the group consisting of moieties derived from benzene, naphthalene, pyrrole, furane, thiophene, thiazole, isothiazole, oxazole, isooxazole, pyrazole, imidazole, 1,2,3-oxadiazole, 1,2,4-oxadiazole, 1,2,5-oxadiazole, 1,3,4-oxadiazole, 1,2,3-triazole, 1,2,4-triazole, pyridine, pyridazine, pyrimidine, pyrazine, 1,2,4-triazine, 1,3,5-triazine, preferably optionally substituted benzene.

A particularly preferred embodiment of the invention relates to compounds of formula (IV), (V), and (VI):

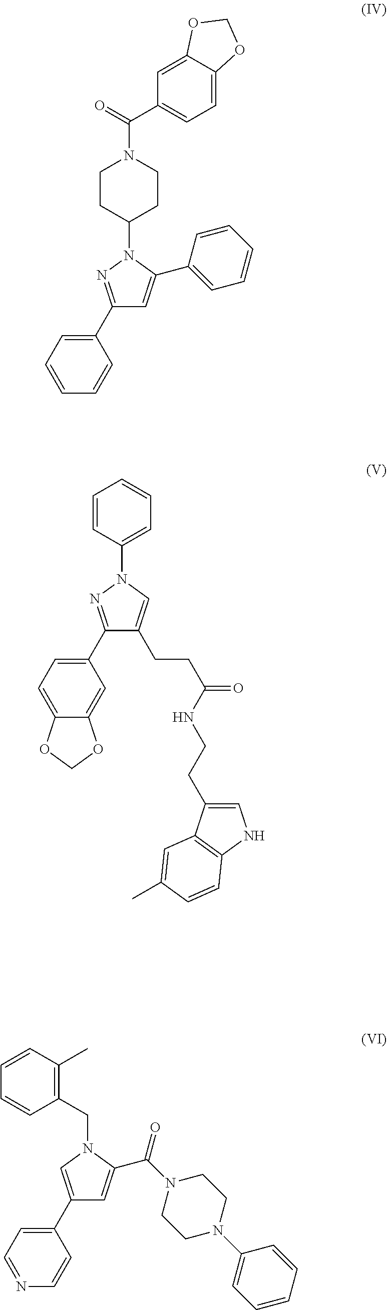 [SMILES: O=C(c1ccc2c(c1)OCO2)N1CCC(n2nc(-c3ccccc3)cc2-c2ccccc2)CC1 ; Cc1ccc2[nH]cc(CCNC(=O)CCc3cn(-c4ccccc4)nc3-c3ccc4c(c3)OCO4)c2c1 ; Cc1ccccc1Cn1cc(-c2ccncc2)cc1C(=O)N1CCN(c2ccccc2)CC1]

or any pharmaceutically acceptable salts or solvates thereof. Particularly preferred are compounds (IV) and (V), and most preferred is compound (IV).

A second aspect of the invention relates to a compound in accordance with the first aspect of the invention, i.e. a compound of any of formula (I) to (VI) for use as a medicament.

A third aspect of the invention relates to a compound in accordance with the first aspect of the invention for use in the treatment of cancer. Preferably, said cancers may be cancers related to the Wnt-signalling pathway, such as cancers relying on the Wnt-signalling pathway, including cancers which may be treated or prevented by inhibition of the Wnt-signalling pathway.

In an embodiment of the invention, the treated cancer is selected from the group consisting of Gliomas (e.g. glioblastomas, astrocytomas), leukemias (e.g. Acute Lymphoblastic Leukemia (ALL), Acute Myeloid Leukemia (AML), Chronic Lymphocytic Leukemia (CLL), Chronic Myelogenous Leukemia (CML)), Adrenocortical Carcinoma, Skin Cancer (e.g. Basal Cell Carcinoma of the Skin, Squamous Cell Carcinoma of the Skin, Melanoma), Bile Duct Cancer (Cholangiocarcinoma), Bladder Cancer (e.g. Ewing Sarcoma, Osteosarcoma, Chondrosarcoma), Breast Cancer, Triple negative breast cancer (TNBC), Colorectal Cancer, Craniopharyngioma, Endometrial Cancer, Ependymoma, Esophageal Cancer, Gastric (Stomach) Cancer, Gastrointestinal Carcinoid Tumor, Hepatocellular (Liver) Cancer, Intraocular Melanoma, Islet Cell Tumors, Renal Cancer (including Wilms Tumor), Laryngeal Cancer, Lip and Oral Cavity (Mouth) Cancer, Non-Small Cell Lung Cancer, Lymphoma (B-cell, Hodgkin), Mesothelioma, Myeloma (e.g. Multiple Myeloma/Plasma Cell Neoplasms, Myelodysplastic Syndromes, Myelodysplastic/Myeloproliferative Neoplasms), Nasopharyngeal Cancer, Neuroblastoma, Ovarian Cancer, Pancreatic Cancer, Pituitary Tumor, Prostate Cancer, Rhabdomyosarcoma, Skin Cancer, Testicular Cancer, Thyroid Cancer, Cervical cancer, Embryonal Tumors;

Atypical Teratoid/Rhabdoid Tumor, Carcinoid Tumor (Gastrointestinal), Germ Cell Tumor, Gastrointestinal Stromal Tumors (GIST) (Soft Tissue Sarcoma), Histiocytosis (Langerhans Cell), Langerhans Cell Histiocytosis, Parathyroid Cancer, Penile Cancer, Pharyngeal Cancer, Retinoblastoma, Uterine Cancer, AIDS-Related Cancers such as Kaposi Sarcoma (Soft Tissue Sarcoma), Non-Hodgkin Lymphoma, Anal Cancer, Cutaneous T-Cell Lymphoma, Fallopian Tube Cancer, Gallbladder Cancer, Salivary Gland Cancer, Papillomatosis.

Primary CNS Lymphoma (Lymphoma), Appendix cancer, Bronchial Tumors, Cardiac (Heart) Tumors, Chordoma, Esthesioneuroblastoma, Gestational Trophoblastic Disease, Hairy Cell Leukemia, Hypopharyngeal Cancer, Metastatic Squamous Neck Cancer with Occult Primary, Midline Tract Carcinoma Involving NUT Gene, Mycosis Fungoides, Nasal Cavity and Paranasal Sinus Cancer, Pancreatic Neuroendocrine Tumors (Islet Cell Tumors), Pleuropulmonary Blastoma, Primary Peritoneal Cancer, Childhood Vascular Tumors, Small Cell Lung Cancer, Oropharyngeal Cancer and Hypopharyngeal Cancer, Thymoma and Thymic Carcinoma, Transitional Cell Cancer of the Renal Pelvis and Ureter Ureter and Renal Pelvis (Transitional Cell Cancer), Urethral Cancer, Vaginal Cancer, Vascular Tumors, Vulvar Cancer, Merkel Cell Carcinoma.

Certain cancer types are presently considered to be related to the Wnt pathway, and thus may be affected by inhibition of this pathway. Thus, a preferred embodiment of the invention is the treatment of a cancer selected from the group consisting of Gliomas (e.g. glioblastomas, astrocytomas), leukemias (e.g. Acute Lymphoblastic Leukemia (ALL), Acute Myeloid Leukemia (AML), Chronic Lymphocytic Leukemia (CLL), Chronic Myelogenous Leukemia (CML)), Adrenocortical Carcinoma, Skin Cancer (e.g. Basal Cell Carcinoma of the Skin, Squamous Cell Carcinoma of the Skin, Melanoma), Bile Duct Cancer (Cholangiocarcinoma), Bladder Cancer (e.g. Ewing Sarcoma, Osteosarcoma, Chondrosarcoma), Breast Cancer, Triple negative breast cancer (TNBC), Colorectal Cancer, Craniopharyngioma, Endometrial Cancer, Ependymoma, Esophageal Cancer, Gastric (Stomach) Cancer, Gastrointestinal Carcinoid Tumor, Hepatocellular (Liver) Cancer, Intraocular Melanoma, Islet Cell Tumors, Renal Cancer (including Wilms Tumor), Laryngeal Cancer, Lip and Oral Cavity (Mouth) Cancer, Non-Small Cell Lung Cancer, Lymphoma (B-cell, Hodgkin), Mesothelioma, Myeloma (e.g. Multiple Myeloma/Plasma Cell Neoplasms, Myelodysplastic Syndromes, Myelodysplastic/Myeloproliferative Neoplasms), Nasopharyngeal Cancer, Neuroblastoma, Ovarian Cancer, Pancreatic Cancer, Pituitary Tumor, Prostate Cancer, Rhabdomyosarcoma, Skin Cancer, Testicular Cancer, Thyroid Cancer, Cervical cancer, Embryonal Tumors;

Atypical Teratoid/Rhabdoid Tumor, Carcinoid Tumor (Gastrointestinal), Germ Cell Tumor, Gastrointestinal Stromal Tumors (GIST) (Soft Tissue Sarcoma), Histiocytosis (Langerhans Cell), Langerhans Cell Histiocytosis, Parathyroid Cancer, Penile Cancer, Pharyngeal Cancer, Retinoblastoma, Uterine Cancer, AIDS-Related Cancers such as Kaposi Sarcoma (Soft Tissue Sarcoma), Non-Hodgkin Lymphoma, Anal Cancer, Cutaneous T-Cell Lymphoma, Fallopian Tube Cancer, Gallbladder Cancer, Salivary Gland Cancer, Papillomatosis.

A more preferred embodiment of the invention is the treatment of a cancer selected from the group consisting of Gliomas (e.g. glioblastomas, astrocytomas), leukemias (e.g. Acute Lymphoblastic Leukemia (ALL), Acute Myeloid Leukemia (AML), Chronic Lymphocytic Leukemia (CLL), Chronic Myelogenous Leukemia (CML)), Adrenocortical Carcinoma, Skin Cancer (e.g. Basal Cell Carcinoma of the Skin, Squamous Cell Carcinoma of the Skin, Melanoma), Bile Duct Cancer (Cholangiocarcinoma), Bladder Cancer (e.g. Ewing Sarcoma, Osteosarcoma, Chondrosarcoma), Breast Cancer, Triple negative breast cancer (TNBC), Colorectal Cancer, Craniopharyngioma, Endometrial Cancer, Ependymoma, Esophageal Cancer, Gastric (Stomach) Cancer, Gastrointestinal Carcinoid Tumor, Hepatocellular (Liver) Cancer, Intraocular Melanoma, Islet Cell Tumors, Renal Cancer (including Wilms Tumor), Laryngeal Cancer, Lip and Oral Cavity (Mouth) Cancer, Non-Small Cell Lung Cancer, Lymphoma (B-cell, Hodgkin), Mesothelioma, Myeloma (e.g. Multiple Myeloma/Plasma Cell Neoplasms, Myelodysplastic Syndromes, Myelodysplastic/Myeloproliferative Neoplasms), Nasopharyngeal Cancer, Neuroblastoma, Ovarian Cancer, Pancreatic Cancer, Pituitary Tumor, Prostate Cancer, Rhabdomyosarcoma, Skin Cancer, Testicular Cancer, Thyroid Cancer, Cervical cancer, Embryonal Tumors.

In an embodiment of the invention a compound of the first aspect of the invention is used in the treatment of cancers dependent on the Wnt pathway.

In a preferred embodiment of the invention a compound of the first aspect of the invention is used in the treatment of breast cancer, particularly triple negative breast cancer.

A fourth aspect of the invention relates to a method of treating cancer, such as cancers dependent on the Wnt pathway, preferably triple negative breast cancer, said method involving the step of administering a compound of the first aspect of the invention to a patient in need thereof.

In an embodiment of the invention the compounds according to the invention are administered in an effective amount. By effective amount is meant a dose necessary to obtain a desired clinical effect. Preferably, the dose is chosen such that in vivo concentration is within the therapeutic window, to optimize between efficacy and toxicity, achieving the greatest therapeutic benefit without resulting in unacceptable side-effects or toxicity.

The compound may be administered by any pharmaceutically acceptable route including methods selected from the group consisting of oral administration, intravenous administration, and subcutaneous administration. Oral administration may be in the form of a tablet, sachet or capsule. Intravenous administration and subcutaneous administration may be in the form of a solution, preferable an aqueous solution, most preferably a buffered aqueous solution.

In another embodiment of the invention the compound to the first aspect of the invention is administered in combination with an additional pharmaceutically acceptable anti-cancer compound. In addition to an additive effect, the skilled person are well aware that certain combinations of compounds may lead to synergistic effects (i.e. effects larger than addition of individual effects). This is desirable and may allow for administration of a lower dose of the individual compounds. In yet another embodiment of the invention, said additional pharmaceutically acceptable anti-cancer compound is a compound effective in the treatment of breast cancer, such as triple negative breast cancer.

In a preferred embodiment of the invention, said additional pharmaceutically acceptable anti-cancer compound is selected from the group consisting of Raloxifene Hydrochloride, Tamoxifen Citrate, Abemaciclib, Methotrexate, Paclitaxel Albumin-stabilized Nanoparticle Formulation, Ado-Trastuzumab Emtansine, Everolimus, Anastrozole, Pamidronate Disodium, Exemestane, Capecitabine, Clafen, Cyclophosphamide, Docetaxel, Doxorubicin Hydrochloride, Epirubicin Hydrochloride, Eribulin Mesylate, Everolimus, Exemestane, 5-FU (Fluorouracil Injection), Toremifene, Fulvestrant, Letrozole, Methotrexate, Fulvestrant, Gemcitabine Hydrochloride, Goserelin Acetate, Eribulin Mesylate, Trastuzumab, Palbociclib, Ixabepilone, Ixabepilone, Ado-Trastuzumab Emtansine, Ribociclib, Lapatinib Ditosylate, Letrozole, Megestrol Acetate, Cyclophosphamide, Neratinib Maleate, Tamoxifen Citrate, Paclitaxel, Palbociclib, Pamidronate Disodium, Pertuzumab, Ribociclib, Docetaxel, Thiotepa, Toremifene, Trastuzumab, Lapatinib Ditosylate, Vinblastine Sulfate, Abemaciclib, Capecitabine, Goserelin Acetate. In another embodiment of the invention the compound to the first aspect of the invention is administered in combination with several additional pharmaceutically acceptable anti-cancer compounds such as two to three additional compounds. Compounds used in such combination therapies may be administered simultaneously or in staggered regimes.

A fifth aspect of the invention relates to a composition comprising the compound according to first aspect and a pharmaceutically acceptable carrier or excipient. The term pharmaceutically acceptable carrier or excipient has the usual meaning in the art and refers to any additive used in the formulation such as a filler, binder, disintegrant, lubricant, solvent, buffers, dispersant or coating necessary to prepare the formulation. The dosage form may be any dosage form well known to the skilled person in the art such as a tablet, sachet, capsule, suspension, solution, cream, emulsion, gel, liposome, or an ointment.

A sixth aspect of the invention relates to a composition comprising the compound according to the first aspect, an additional pharmaceutically acceptable anti-cancer compound, and a pharmaceutically acceptable carrier or excipient. Any carrier, excipient or pharmaceutically acceptable anti-cancer compound mentioned above is suitable.

Another embodiment of the invention relates to a composition comprising a compound according to the first aspect of the invention, wherein said additional pharmaceutically acceptable anti-cancer compound is a compound effective in the treatment of breast cancer, such as triple negative breast cancer.

It should be noted that embodiments and features described in the context of one of the aspects of the present invention also apply to the other aspects of the invention. Particularly, the embodiments relating to the compounds, also apply to the same compounds for use as a medicament and for use in the treatment of the cancers.

All patent and non-patent references cited in the present application, are hereby incorporated by reference in their entirety.

The invention will now be described in further details in the following non-limiting examples.

### EXAMPLES

### Materials and Methods

### General

If not stated otherwise, cells were grown in DMEM 10% FBS, 1% PenStrep (Gibco). Cells were incubated at 37° C., 5% CO₂, >80% RH. The triple negative cell lines (ATCC) used are: BT-20, HCC 1395, MDA-MB 231, MDA-MB 468 and HCC 1806. For β-catenin stabilization assays and Western Blot analysis, mouse L-cells were also used.

### Compound Identification

A commercial (ChemDiv Inc., San Diego, Calif., US) small molecule library (high-diversity GPCR-targeted compound library containing 1000 compounds selected by ChemDiv focusing on maximizing chemical diversity from the 40′000 GPCR-targeted compound library [see http://www.chemdiv.com, particularly http://www.chemdiv.com/gpcr-target-platform-library-2/]) containing 1000 compounds was screened for Wnt pathway inhibitory effect using the TOPflash assay. This screen identified compound 1 (FSA) as the most potent inhibitor of the Wnt pathway in vitro. This compound was selected for further development. In a first round of screening, 34 randomly chosen compounds resembling compound 1, but presenting some intravariance were tested for their ability to inhibit the Wnt pathway, using the TOPFLash reporter assay. Compounds were first tested at concentrations of 5 and 50 μM to show concentration dependency. The IC₅₀ values and efficacy of the most promising compounds were then determined for aiding with the selection of the second round of compounds to be screened.

For the second screen the compounds were selected by overall chemical (Tanimoto) and substructural (via generalized structure search) similarity in the ChemDiv collection (ca. 1.5 mio compounds). The overall set of similars (ca. 1000 compounds) was clustered using the JChem software and 1-2 representatives of each substructural cluster were selected for analysis, resulting in the comprehensive list of 117 compounds.

### TopFlash Assay

For the screens, BT-20 cells, stably transfected with the TOPFlash reporter plasmid were seeded at 15K cells per well in white tissue-culture-treated 96-well plates (Greiner) and incubated overnight. If needed, the cells were additionally transfected with the pRL-CMV plasmid using the X-tremeGENE HP DNA transfection reagent (Roche) according to the manufacturer's protocol and again incubated overnight. The cells were pretreated for 1 h with DMSO or compound before addition of Wnt3a (250 ng/ml final concentration) and incubation for 18-24 h. The medium was then removed and 12 μl 10% sucrose solution was added to the cells to prevent drying. The plates were then read using the Victor3 Multilabel Counter (PerkinElmer) after injection of the luciferase firefly buffer (50 μl; 25 mM glycylglycine, 15 mM K×PO₄, 4 mM EGTA, 2 mM ATP, 1 mM DTT, 15 mM MgSO₄, 0.1 mM CoA, 75 μM luciferin, pH 8.0) together with the lysis buffer (15 μl; 25 mM glycylglycine pH 7.8, 1% Triton X-100, 15 mM MgSO₄, 4 mM EGTA, 1 mM DTT) followed by the renilla firefly buffer (50 μl; 1.1 M NaCl, 2.2 mM Na2EDTA, 0.22 M K×PO₄, 0.44 mg/mL BSA, 1.3 mM NaN₃, 1.43 μM coelenterazine, pH 5.0). Data was analyzed using the Prism 6 Software (GraphPad).

### Proliferation Assay (MTT)

TNBC cell lines and were seeded at previously determined concentrations in 96-well plates and incubated for 24 h. The next day, the medium was replaced with medium containing either compound or the respective amount of DMSO for the controls. The proliferation was measured after 72 h, by addition of a solution of 1 mg/ml thiazolyl blue (Roth) in PBS, further incubation for 2 h-4 h at 37° C., and lysis of the cells by addition of 50 μl DMSO. The absorbance was read at 570 nm using the Victor3 Multilabel Counter (PerkinElmer).

### Migration Assay

Cellular migration was measured by using the so-called scratch-wound assay. TNBC cell lines were seeded to confluency in clear flat-bottom 96-well plates and incubated over-night. The following day a straight wound was inflicted on the monolayer using a 10 μl pipette tip. The cells were then washed carefully with PBS and treated with media containing the compounds or DMSO. Each well was imaged individually and the cells incubated for 6-18 h. Following this, the wells were again imaged and the migration of the cell front was measured using ImageJ.

### Colony Forming Assay

TNBC cell lines were seeded at previously determined concentrations in 6-well plates and incubated for 24 h. The cells were then treated with the compounds or DMSO only and the colony formation followed daily by visual inspection. Once the colonies were big enough (70-100 cells), the cells were fixed with a solution of 4% PFA in PBS pH 7.4. The colonies were then stained using a solution of 1% Crystal violet and images taken of the individual wells to count the number of colonies. The colony counting and analysis was done using ImageJ.

### β-Catenin Stabilization Assay and Immunoblotting

Cells were seeded to 70-80% confluency in 12-well plates and incubated overnight. The medium was then changed for medium containing compound or DMSO and the cells pre-incubated for 1 h. Wnt3a for Wnt pathway stimulation was added directly to a final concentration of 250 ng/ml and incubated to allow for β-catenin stabilization (L-cells, 6 h; HCC 1395 and BT-20, 18 h), DVL phosphorylation (1.5-2 h) or LRP6 phosphorylation (1.5 h). After washing the cells with ice cold PBS, the cells were lysed by addition of 70 μl RIPA buffer (50 mM Tris pH 7.4, 1% Triton X-100, 0.1% SDS, 150 mM NaCl, 1 mM EDTA, 1 mM DTT, protease inhibitors (Roche)) containing phosphatase inhibitors if needed (4 mM NaF, 4 mM Imidazole, 2.3 mM Na₂MoO₄, 4 mM Na₃VO₄, 8 mM M C₄H₄Na₂O₆*2H₂O, 2 mM Na₄P₂O₇, 2 mM β-Glycerophosphate) and shaking 10 min on ice. The cell lysate was collected and centrifuged for 15 min at 16000 g at 4° C. to remove cell debris. The samples were equilibrated using the Bradford method and further separated and analyzed by SDS-PAGE and Western blot respectively. The following antibodies were used: anti β-catenin, 1:1000, BD Bioscience #610153; Anti active β-catenin, 1:1000, Merck Millipore #05-665; anti DVL2, 1:1000, Cell Signaling #3223S; anti DVL3, 1:1000, Cell Signaling #3218S; anti p-LRP6 (S1490), 1:1000, Cell Signaling #2568S; anti a-tubulin, 1:2000, Sigma #T6199.

### Example 1: Potency and Efficacy of Identified Wnt Pathway Inhibitors Using the TOPFlash Reporter Assay

TABLE 1
Average Potency IC₅₀ (μM) and Efficacy (%).

| | | | |
|---|---|---|---|
| Compound no. | | | |
| (internal ref.) | Catalogue no.* | IC₅₀ (μM) | Efficacy (%) |

| | | | | |
|---|---|---|---|---|
| 1 | (FSA) | F368-0488 | 11.0 | 86.3 |
| 2 | (16) | E136-1056 | 16.4 | 46.8 |
| 3 | (18) | 5237-1505 | >100 | 29.7 |
| 4 | (19) | 3935-0561 | 22.6 | 92.5 |
| 5 | (32) | F368-0051 | 10.3 | 86.7 |
| 6 | (33) | F368-0459 | 17.5 | 79.4 |
| 7 | (34) | F368-0520 | 15.2 | 85.0 |
| 8 | (38) | F368-0269 | 29.2 | 74.9 |
| 9 | (39) | F368-0446 | 21.9 | 50.8 |
| 10 | (42) | F368-0052 | 5.1 | 82.9 |
| 11 | (43) | F368-0417 | 5.2 | 88.4 |
| 12 | (47) | F368-0486 | 15.1 | 34.8 |
| 13 | (48) | F368-0519 | 6.5 | 77.0 |
| 14 | (50) | F368-0395 | 47.1 | 74.9 |
| 15 | (51) | F368-0435 | 7.5 | 83.4 |
| 16 | (55) | F368-0516 | 17.4 | 67.1 |
| 17 | (58) | F368-0350 | 25.1 | 78.6 |
| 18 | (59) | F368-0031 | 7.9 | 63.0 |
| 19 | (60) | F368-0831 | 11.5 | 64.0 |
| 20 | (61) | F368-0371 | 7.2 | 91.7 |
| 21 | (62) | F368-0924 | 80.6 | 65.0 |
| 22 | (67) | F368-0277 | 53.6 | 53.4 |
| 23 | (69) | F368-0510 | 27.9 | 74.6 |
| 24 | (96, F2-95) | P076-0599 | 18.3 | 94.8 |
| 25 | (106, F2-99) | V006-4971 | 24.1 | 85.2 |
| 26 | (108) | V012-3790 | 16.5 | 72.7 |
| 27 | (109) | V008-3606 | 14.3 | 86.5 |

*ChemDiv library catalogue number

### Example 2: Structure of Identified Wnt Pathway Inhibitors

TABLE 2
Structure of compounds 1-27.

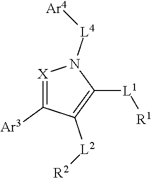

| Nr. | L¹ | L² | L⁴ | R¹ |
|---|---|---|---|---|
| 1 | bond | 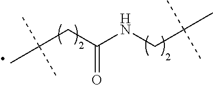 | bond | H |
| 2 | bond | 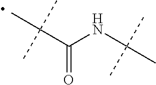 | bond | H |
| 3 | bond | 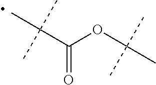 | bond | H |
| 4 | bond | 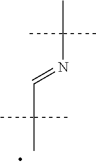 | bond | H |
| 5 | bond | 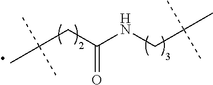 | bond | H |
| 6 | bond | 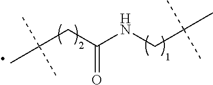 | bond | H |
| 7 | bond | 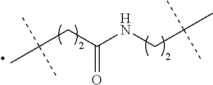 | bond | H |
| 8 | bond | 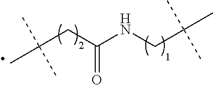 | bond | H |
| 9 | bond | 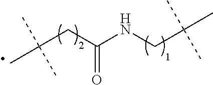 | bond | H |
| 10 | bond | 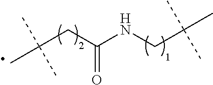 | bond | H |
| 11 | bond | 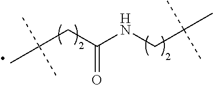 | bond | H |
| 12 | bond | 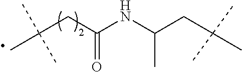 | bond | H |
| 13 | bond | 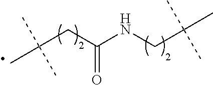 | bond | H |
| 14 | bond | 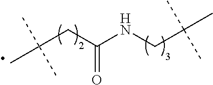 | bond | H |
| 15 | bond | 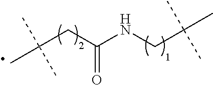 | bond | H |
| 16 | bond | 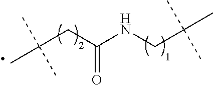 | bond | H |
| 17 | bond | 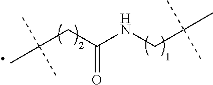 | bond | H |
| 18 | bond | 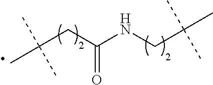 | bond | H |
| 19 | bond | 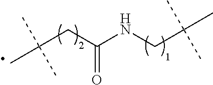 | bond | H |
| 20 | bond | 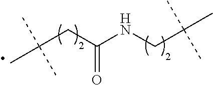 | bond | H |
| 21 | bond | 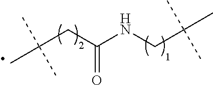 | bond | H |
| 22 | bond | 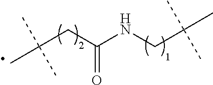 | bond | H |
| 23 | bond | 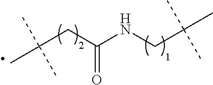 | bond | H |
| 24 | 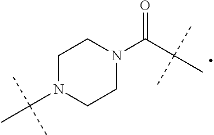 | bond | —CH₂— | 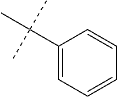 |
| 25 | bond | bond | 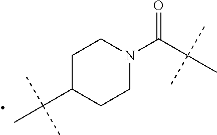 | 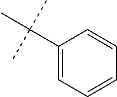 |
| 26 | bond | bond | 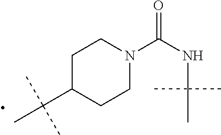 | 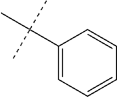 |
| 27 | bond | bond | 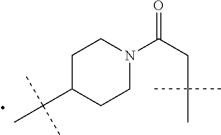 | 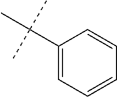 |

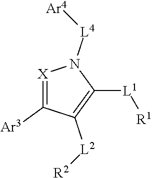

| Nr. | R² | Ar³ | Ar⁴ | X |
|---|---|---|---|---|
| 1 | 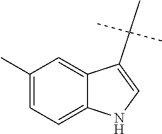 | 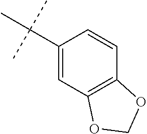 | 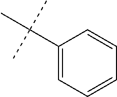 | N |
| 2 | 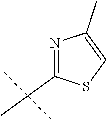 | 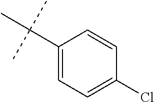 | 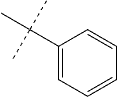 | N |
| 3 | 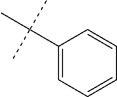 | 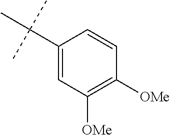 | 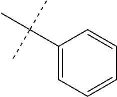 | N |
| 4 | 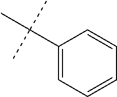 | 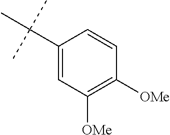 | 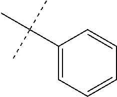 | N |
| 5 | 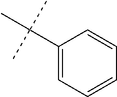 | 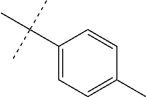 | 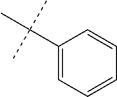 | N |
| 6 | 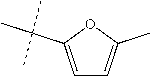 | 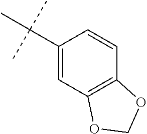 | 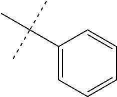 | N |
| 7 | 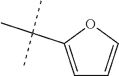 | 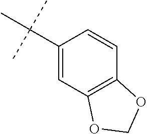 | 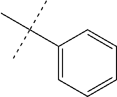 | N |
| 8 | 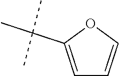 | 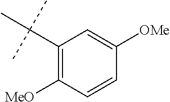 | 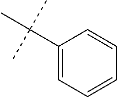 | N |
| 9 | 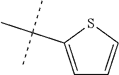 | 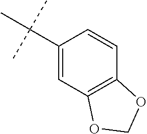 | 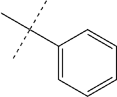 | N |
| 10 | 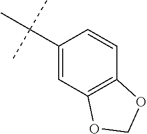 | 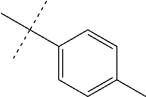 | 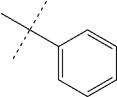 | N |
| 11 | 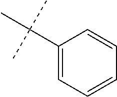 | 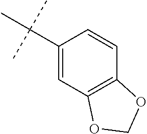 | 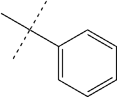 | N |
| 12 | 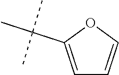 | 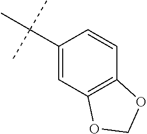 | 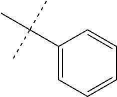 | N |
| 13 | 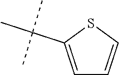 | 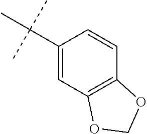 | 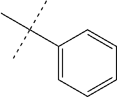 | N |
| 14 | 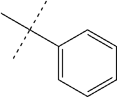 | 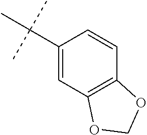 | 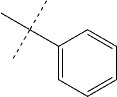 | N |
| 15 | 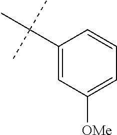 | 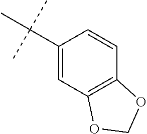 | 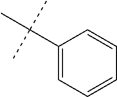 | N |
| 16 | 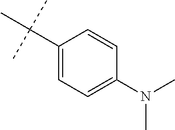 | 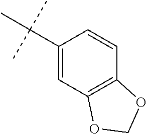 | 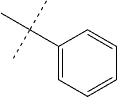 | N |
| 17 | 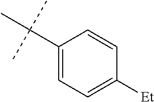 | 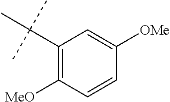 | 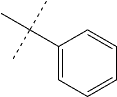 | N |
| 18 | 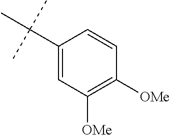 | 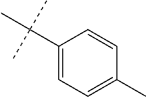 | 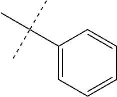 | N |
| 19 | 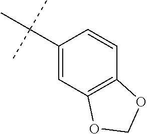 | 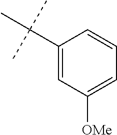 | 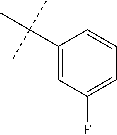 | N |
| 20 | 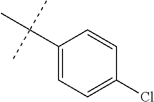 | 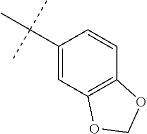 | 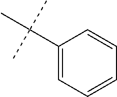 | N |
| 21 | 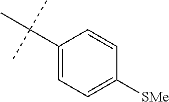 | 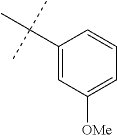 | 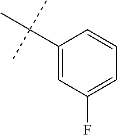 | N |
| 22 | 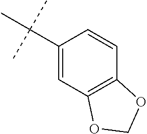 | 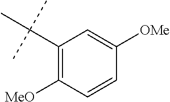 | 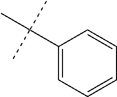 | N |
| 23 | 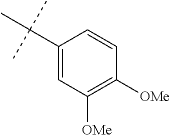 | 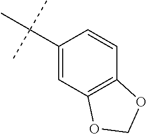 | 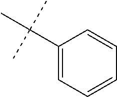 | N |
| 24 | H | 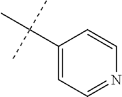 | 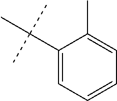 | C H |
| 25 | H | 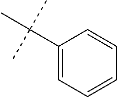 | 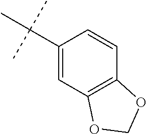 | N |
| 26 | H | 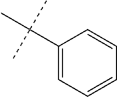 | 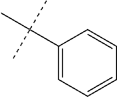 | N |
| 27 | H | 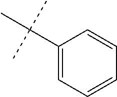 | 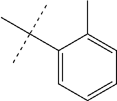 | N |

The dot (•) in the linkers, L¹, L² and L⁴ denotes the end attached to the core scaffold (pyrazole or pyrole).

### Example 3: Wnt Response (% of Control) of Compound 1 Using Either Wnt3a or LiCl for Activation

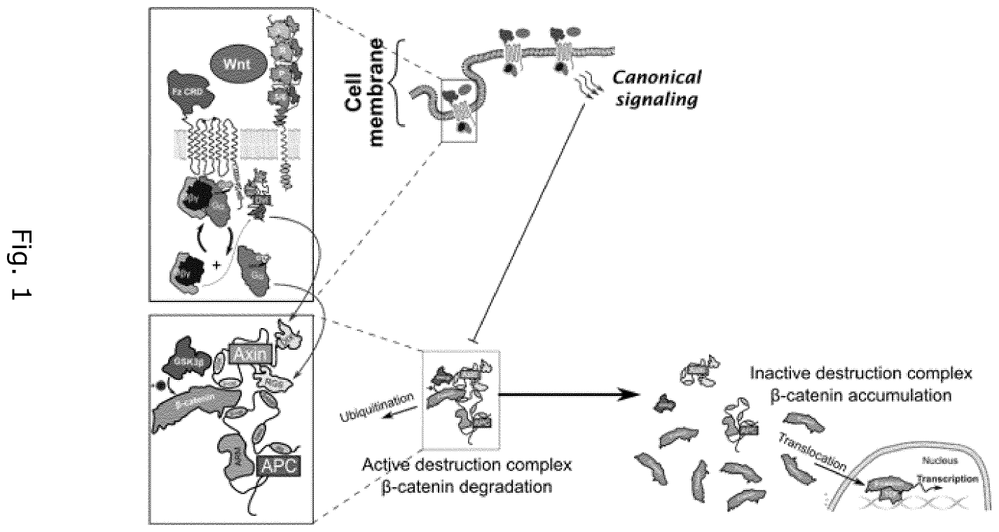

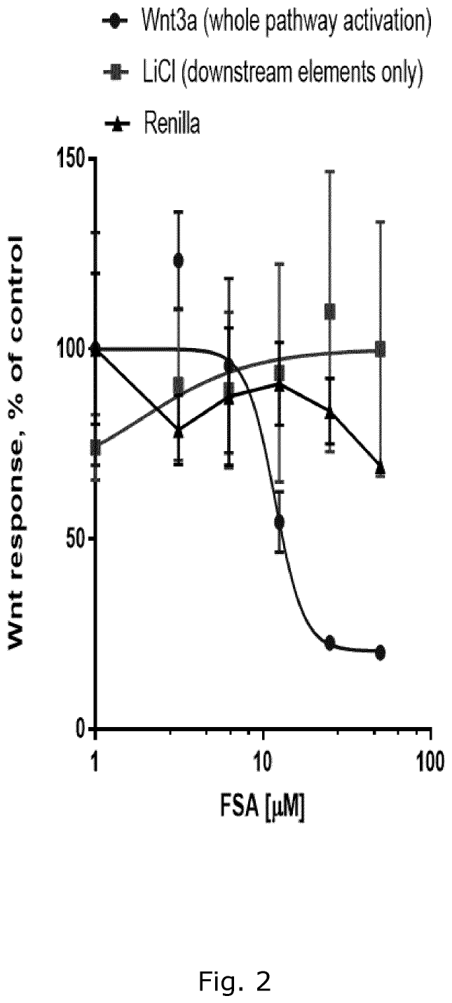

FIG. 2 shows, that compound 1 is efficient in specific suppression of the Wnt3a-stimulated pathway activation, while not changing the levels of Renilla luciferase expressed under control of CMV promoter, thus serving the control of cell well-being. Moreover, when the downstream part of the pathway is activated by LiCl compound 1 do not inhibit confirming that compound 1 must act above the destruction complex of the Wnt pathway (see FIG. 1).

### Example 4: β-Catenin Stabilization Assay

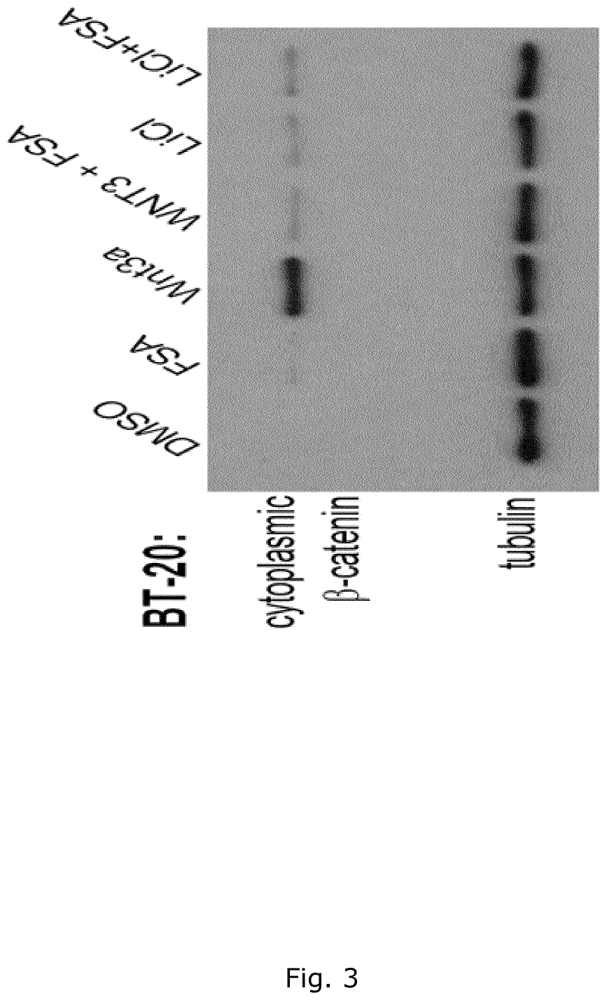

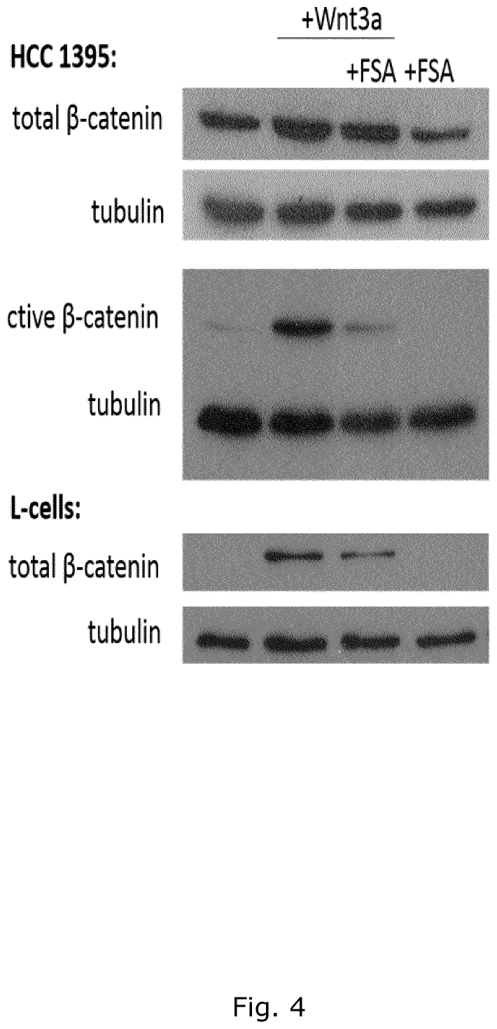

To independently confirm the Wnt inhibitory effects of compound 1, the classical β-catenin stabilisation assay was used (see FIGS. 3 and 4). The effects of compound 1 was analysed on accumulation of cytoplasmic β-catenin in BT-20 cells (see FIG. 3), essentially recapitulating the results obtained using TopFlash.

Compound 1 demonstrates activity in broad spectrum of cell lines (see FIG. 4): L-cells (mouse fibroblasts) have nearly absent basal β-catenin levels and were therefore chosen for this assay; in addition HCC 1395 cells was used as another representative TNBC cell line. The results show a clear decrease of the total μ-catenin levels by FSA in L-cells and a decrease in the active-β catenin levels when tested on HCC 1395 cells, confirming the inhibition of the canonical Wnt/β-catenin pathway (see FIG. 1).

### Example 5: Effect of Compound 1 on the Phosphorylation of DVL

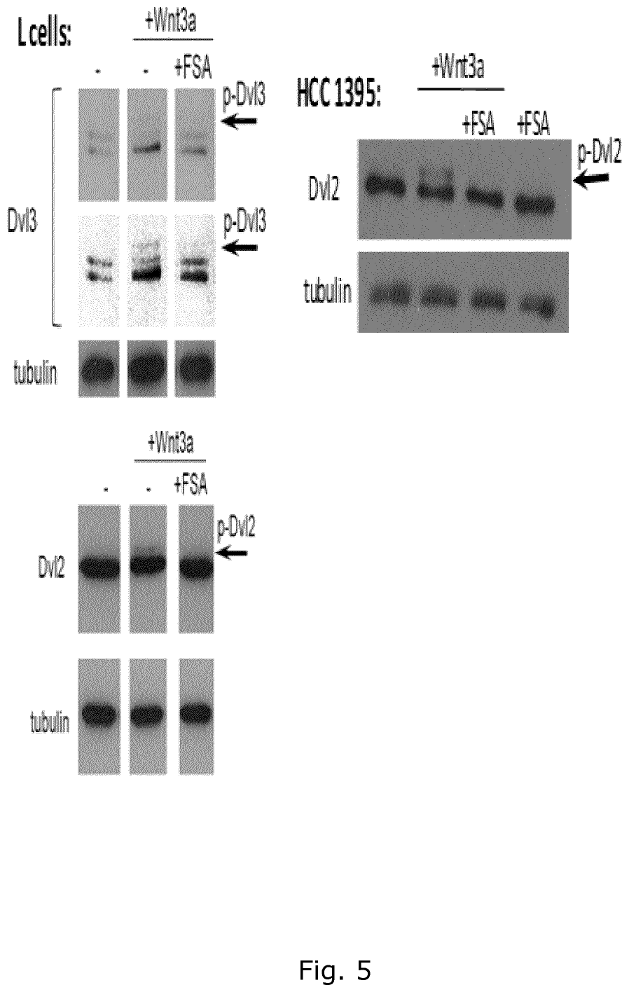

The effects of compound 1 on the upper-level phosphoprotein DVL was examined. The phosphoprotein DVL is phosphorylated on over 40 sites upon Wnt-pathway activation, which can be detected as a shift when analysed by electrophoresis. Compound 1 clearly inhibited the shift observed upon Wnt3a stimulation of L-cells (DVL2 and DVL3) and HCC 1395 cells (DVL2) (see FIG. 5). This indicates that compound 1 targets either DVL itself or a protein upstream of it (see FIG. 1).

### Example 6: Proliferation of TNBC Cell Lines in the Presence of Compound 1 Using the MTT Assay

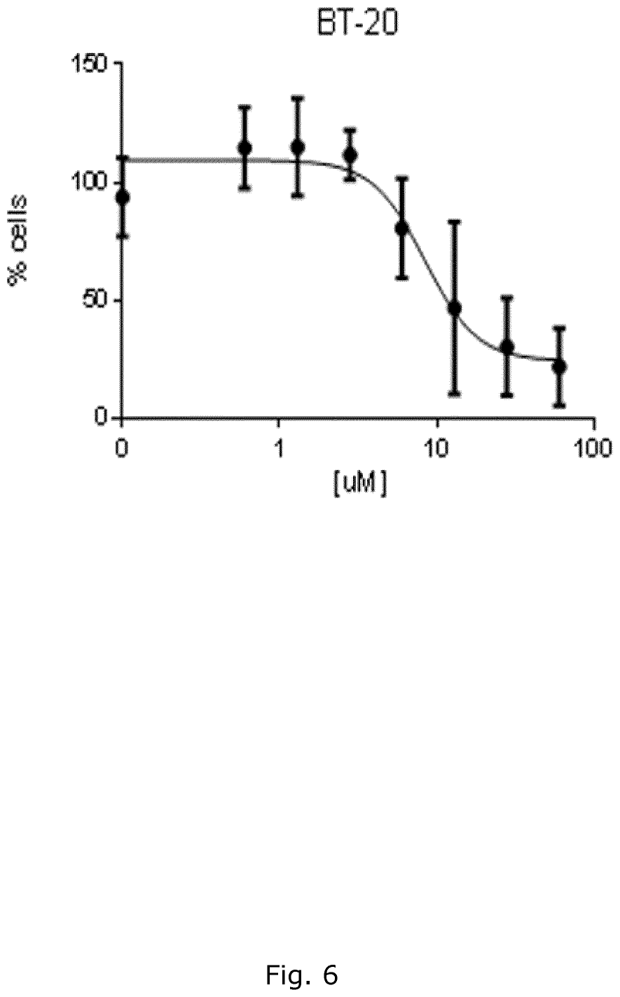

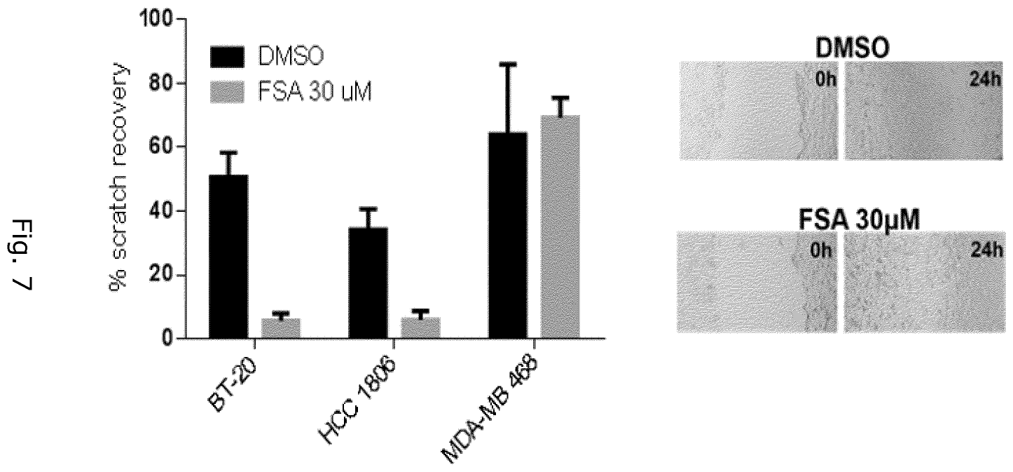

The effect of compound 1 on the proliferation of TNBC cells (BT-20, HCC 1395, MDA-MB 468, HCC 1806, MDA-MB 231) was measured (see FIG. 6 and Table 3). The MTT assay, demonstrated that compound 1 are indeed able to halt the proliferation of the selected cell lines in a concentration dependent manner with an IC₅₀ similar to the IC₅₀ of the Wnt inhibition for BT-20. This indicates that the cell proliferation might be linked to the inhibition of the Wnt pathway and not due to general toxicity. This in vitro data suggest strong anti-cancer properties of compound 1.

TABLE 3
Potency and efficiency for TNBC cell lines
MTT assay with compound 1

| Cell line | IC₅₀ (μM) | Efficiency (%) |
|---|---|---|
| BT-20 | 15 | 90 |
| HCC 1395 | 8 | 90 |
| MDA-MB 468 | 12 | 93 |
| HCC 1806 | 18 | 79 |
| MDA-MB 231 | 6 | 63 |

### Example 7: Migration Assay of TNBC Cell Lines

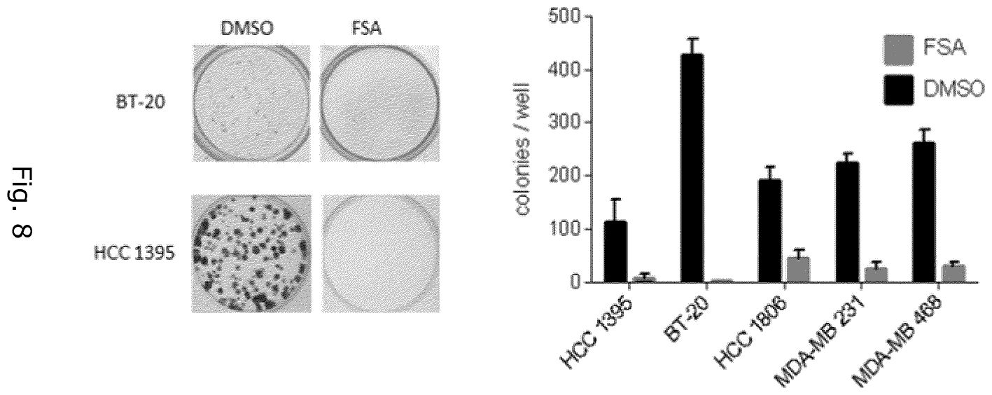

FIG. 7 shows the scratch recovery for the three TNBC cells lines BT-20, HCC 1806 and MDA-MB 468. As can be seen compound 1 clearly inhibits migration of cells except for MDA-MB 468. This in vitro data suggest strong anti-cancer properties of compound 1.

### Example 8: Colony Forming Assay of TNBC Cell Lines

FIG. 8 clearly shows an inhibition of colony formation of different TNBC cell lines in the presence of compound 1. This in vitro data suggest strong anti-cancer properties of compound 1.

### Example 9: In Vitro Microsomal Stability of Compounds 1, 24 and 25

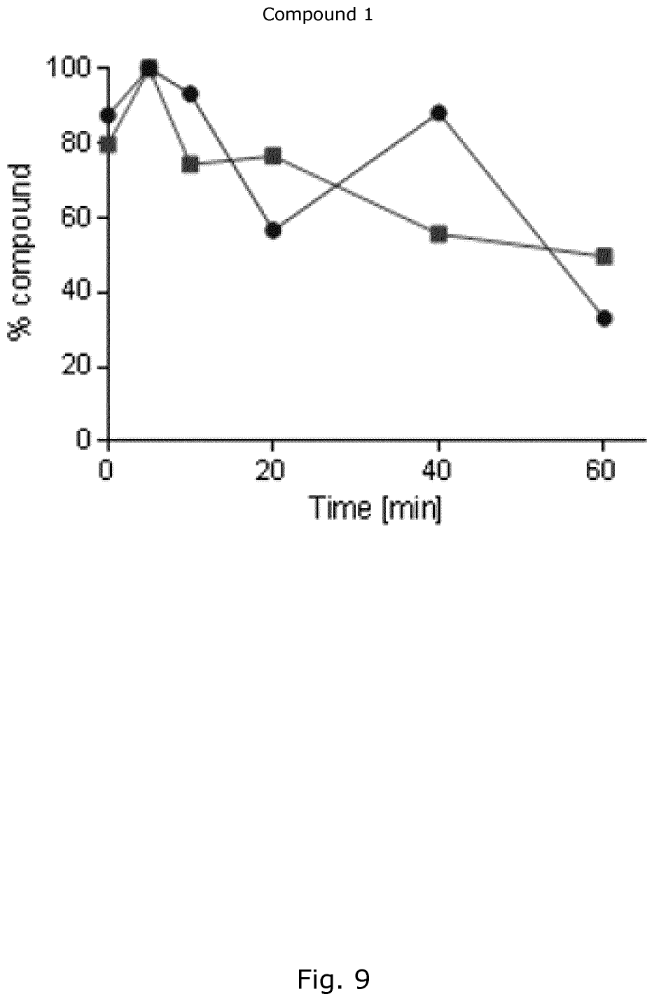

FIG. 9 and Table 4 show the microsomal stability of compounds 1, 24 and 25. These compounds show descent microsomal stability of the compounds in vitro.

TABLE 4
CYP and UGT stability of compound 1, 25 and 26.
Microsomal assay (% compound after 60 min)

| | | |
|---|---|---|
| Compound | CYP stability | CYP UGT stability |

| | | | |
|---|---|---|---|
| 1 | (FSA) | 33 | 49 |
| 24 | (96, F2-95) | 67 | 76 |
| 25 | (106, F2-99) | 60 | 25 |

### Example 10: In Vivo Pharmacokinetic Profiling of Compounds 1, 24 and 25

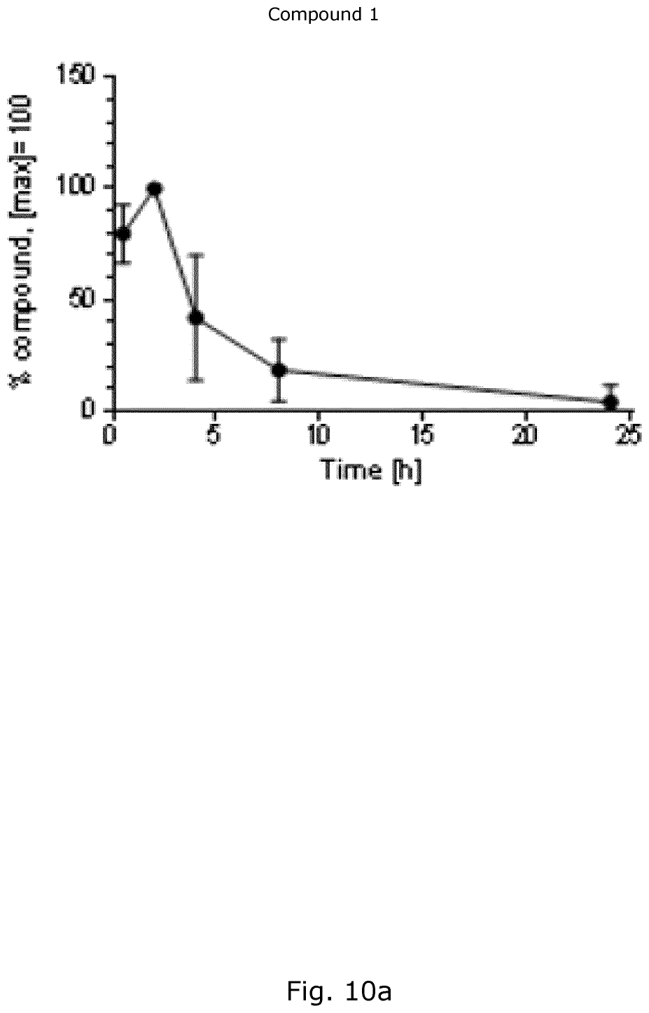

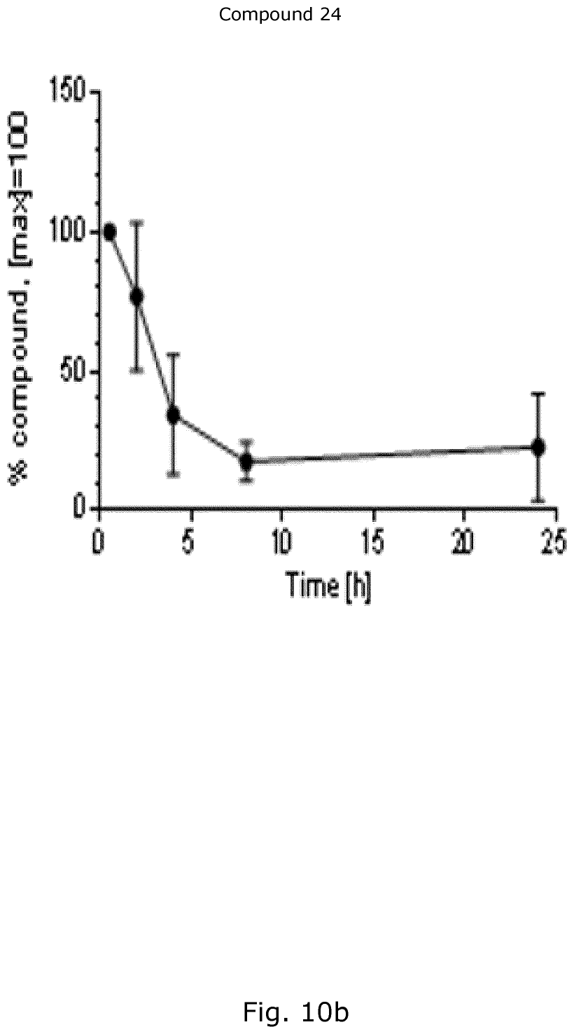

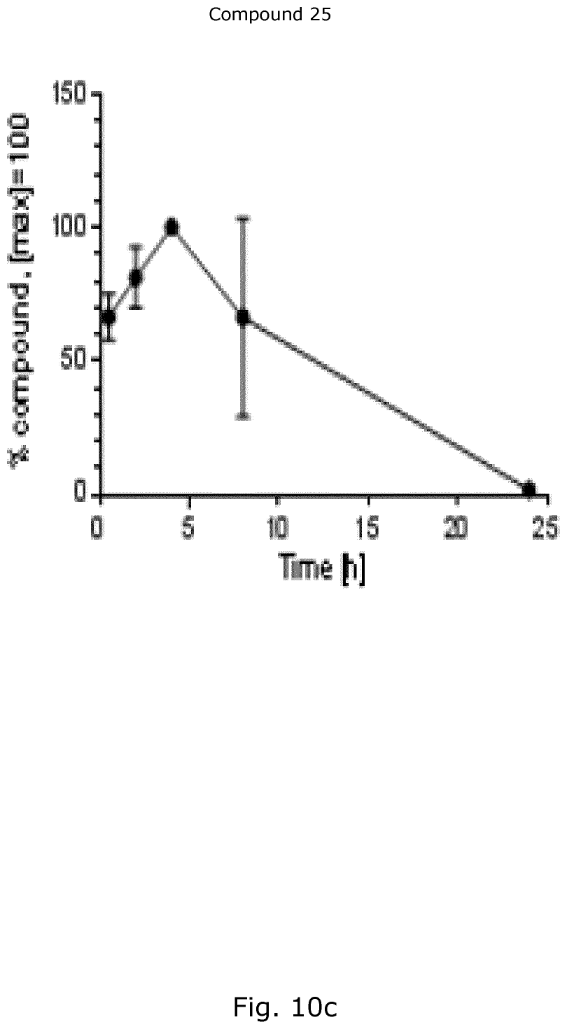

An in vivo experiment was performed to get a rough ADME profile and a first impression of the tolerability of the compounds and to determine any acute toxicity. Three tumour baring mice was injected with the three most promising compounds 1, 24 and 25. After sequentially injecting each of the mice with each of the compounds no overt adverse reactions in animals was observed. Blood samples were taken at regular intervals for studying the kinetics. All compounds had similar elimination profiles with half-lives between 3 and 8 h (see FIG. 10a-c). The tissue analysis (Table 5) showed that compounds 1 and 25 present better tissue levels, i.e. for compound 24 the mean concentration achieved in the breast was around 8 μM while it was around 20 μM for compound 1 and 27 μM for compound 25. The maximal plasma levels achieved were below 1 μM for compound 1 and 24, however were impressively at around 50 μM for compound 25. The compounds showed a general high level of accumulation after only two injections in the breast close to those observed in vitro (20-40 μM). This data is encouraging, as it is hypothesised that high tissue concentrations, will be needed for an in vivo effect on the breast cancer.

TABLE 5
In vivo tissue concentration of compounds 1, 24 and 25.
Tissue concentration [μM]

| Compound | 1 | 24 | 25 |
|---|---|---|---|
| Plasma | 3.9 | 0.3 | 49.9 |
| Tumor | 154.3 | 0.3 | 2219.5 |
| Breast | 92.1 | 8.6 | 728.7 |
| Liver | 136.9 | 0.3 | 1727.8 |
| Lung | 6.2 | 0.3 | 36.3 |
| Intestine | 44.0 | 0.3 | 43.3 |
| Urine | 6.6 | 0.1 | 0.1 |
| Faeces | 0.1 | 0.1 | 0.2 |

### Example 11: Synthesis of Compound 1 (FSA)

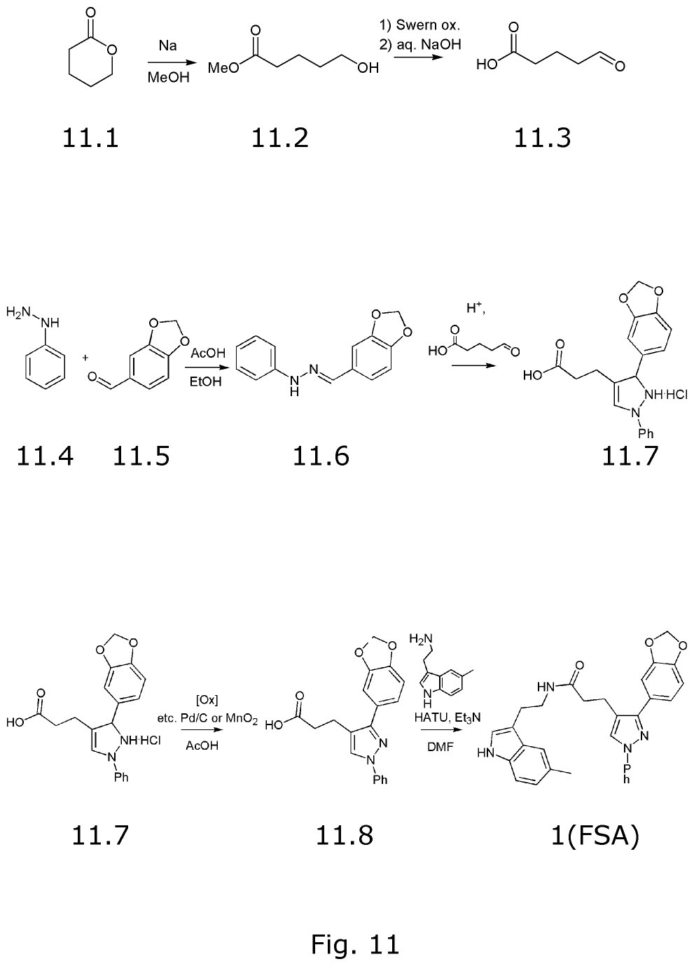

Compound 1 is synthesized in four steps from commercially available starting materials (see FIG. 11). Phenylhydrazine (11.4) (1 eq.) and piperonal (11.5) (1 eq.) is dissolved in anhydrous EtOH and AcOH (0.2 eq.) added. The reaction is stirred at r.t. until completion and the solvent evaporated off to afford crude 11.6. The crude product is redissolved in anhydrous THF followed by addition of HCl (0.5 eq.) and glutaric semialdehyde (1 eq.). The reaction is refluxed under argon atmosphere until completion and the solvent evaporated off to afford crude 11.7. Crude 11.7 is added Pd/C under argon followed by addition of acetic acid. The mixture is stirred at r.t. until completion, filtered through celite and the solvent evaporated off to afford crude 11.8. The crude product was purified by silica gel chromatography to afford pure 11.8. 11.8 is dissolved in anhydrous DMF and Et₃N (5 eq.). HATU (1.05 eq.) is added to the mixture followed by addition of 5-methyltryptamine hydrochloride. The reaction is stirred at r.t. until completion and worked up by addition of EtOAc and sat. aq. NaHCO₃. The phases are separated and the organic phase washed with sat. aq. NaHCO₃(3×), sat. aq. NaCl (1×), dried over Na₂SO₄ and evaporated to dryness to afford crude 1. The crude product was purified by silica gel chromatography to afford 1.

### Example 12: Synthesis of Compound 24 (F2-99)

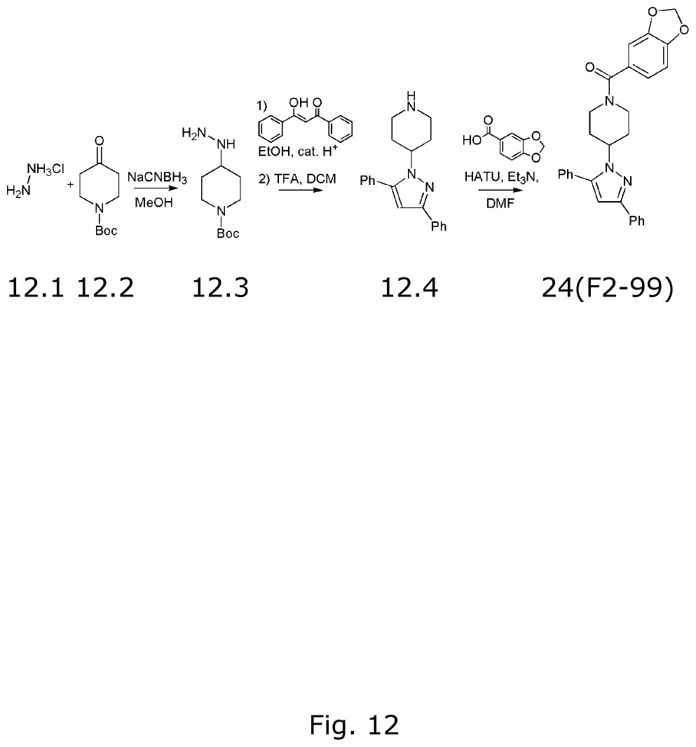

Compound 24 is synthesized in three steps from commercially available starting materials (see FIG. 12). Hydrazine.HCl (20 eq.) (12.1) and 1-Boc-4-piperidone (1 eq.) (12.2) is dissolved in MeOH and NaCNBH₃ (5 eq.) is added and the reaction stirred at r.t. until completion. EtOAc and sat. aq. NaHCO₃ is added and the phases separated. The organic phase is washed with sat. aq. NaHCO₃(3×), sat. aq. NaCl (1×), dried over Na₂SO₄ and evaporated to dryness to afford crude 12.3. The crude product 12.3 is dissolved in anhydrous EtOH and TFA (1 eq.) followed by addition of 3-hydroxy-1,3-diphenyl-propenone. The mixture is refluxed under argon until completion and the solvent evaporated off. The crude product is redissolved in anhydrous DCM and TFA (20 eq.) added. The reaction is stirred at r.t. until complete deprotection. The solvent is evaporated off and EtOAc and sat. aq. NaHCO₃ is added and the phases separated. The organic phase is washed with sat. aq. NaHCO₃(1×), sat. aq. NaCl (1×), dried over Na₂SO₄ and evaporated to dryness to afford crude 12.4. The crude product is purified by silica gel chromatography to afford pure 12.4. 12.4 is dissolved in anhydrous DMF and added to another flask containing piperonylic acid, HATU, Et₃N (5 eq.) dissolved in anhydrous DMF under argon. The reaction was stirred at r.t. until completion. EtOAc and sat. aq. NaHCO₃ was added. The phases were separated and the organic phase washed with sat. aq. NaHCO₃(3×), sat. aq. NaCl (1×), dried over Na₂SO₄ and evaporated to dryness to afford crude 24. The crude product was purified by silica gel chromatography to afford pure 24.

### Example 13: Synthesis of Compound 25 (F2-95)

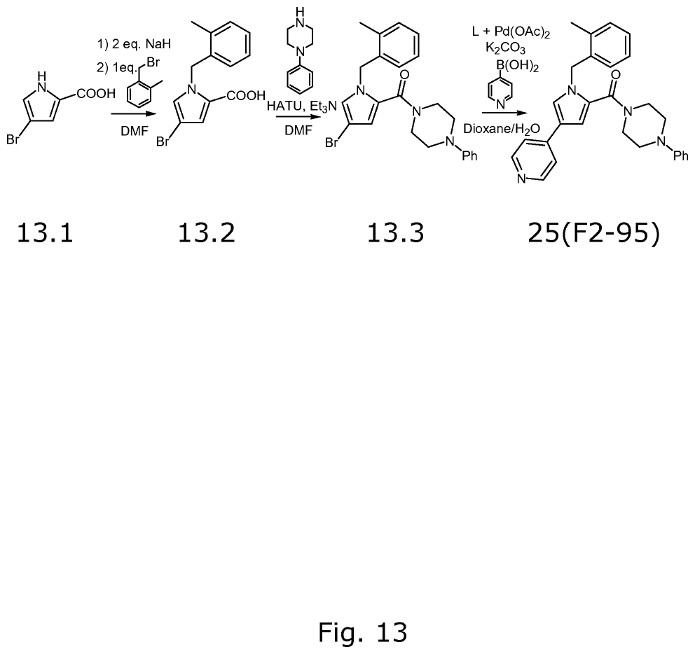

Compound 25 is synthesized in 2 steps from commercially available starting materials (see FIG. 13). 4-Bromopyrrole-2-carboxylic acid is dissolved in anhydrous DMF under argon and cooled to 0° C. followed by slow addition of NaH (2.5 eq.). The reaction is allowed to varm to rt. and stirred until evolution of hydrogen ceases. 2-Methylbenzyl bromide (1.0 eq.) is added dropwise and the reaction stirred at r.t. until completion as judged by TLC. To this reaction mixtures is added Et₃N (5 eq.) and HATU (1.05 eq.) followed 1-phenylpiperazine (1.05 eq.) at r.t. The reaction is stirred for 15 min. and worked up by addition of EtOAc and sat. aq. NaHCO₃. The phases are separated and the organic phase washed with sat. aq. NaHCO₃(3×), sat. aq. NaCl (1×), dried over Na₂SO₄ and evaporated to dryness to afford crude 13.3. The crude product was purified by silica gel chromatography to afford 13.3. 13.3 was dissolved in a mixture of dioxane:H₂O and degassed with argon under ultrasound. To the degassed mixture is added K₂CO₃ (5 eq.), 4-pyridinylboronic acid (1.5 eq.), Pd(OAc)₂ (0.05 eq.) and PPh₃ (0.25 eq.) under argon and the reaction mixture is heated at 80° C. until completion. EtOAc and sat. aq. NaHCO₃ is added and the phases separated and the organic phase washed with sat. aq. NaHCO₃(3×), sat. aq. NaCl (1×), dried over Na₂SO₄ and evaporated to dryness to afford crude 25. The crude product is purified by silica gel chromatography to afford pure 25.

### REFERENCES

1. Nusse, R., Wnt signaling in disease and in development, Cell Res, 2005, 15(1): p. 28-32.
2. Polakis, P., Drugging Wnt signalling in cancer, EMBO J, 2012, 31(12): p. 2737-46.
3. Casás-Selves, M. et al., Target deconvolution of efforts on Wnt pathway screen reveal dual modulation of oxidative phosphorylation and SERCA2, ChemMedChem, 2017, 12: p. 917-924.
4. WO 2008/071398 (Al).
5. Ananda, H. et al., Regioselective synthesis and biological studies of novel 1-aryl-3,5-bis (het) aryl pyrazole derivatives as potential antiproliferative agents, Mol Cell Biochem, 2017, 426 p. 149-160.
6. Madhavilatha, B. et al., Synthesis of 1,2,3-triazole and isoxazole-linked pyrazole hybrids and their cytotoxic activity, Med Chem Res, 2017, 26, p. 1753-1763.

1. A method of treating triple negative breast cancer comprising administering to a subject having triple negative breast cancer, the compound of formula (I): 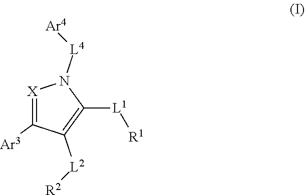
wherein
X is selected from the group consisting of N and CH,
L¹, L², and L⁴ are independently selected from the group consisting of a bond, optionally substituted C₁-C₈ alkylene, optionally substituted C₂-C₈ alkenylene, optionally substituted C₂-C₈ alkynylene, optionally comprising one or more moieties selected from the group consisting of an amide, a thioamide, an ester, an amine, an urea, a carbamate, an aldimine, a ketone and 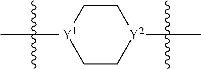
wherein Y¹ and Y² are independently selected from CH and N; or combinations thereof,
R¹ and R² are independently selected from the group consisting of H, optionally substituted aryl and optionally substituted heteroaryl, and
Ar³ and Ar⁴ are independently selected from the group consisting of optionally substituted aryl and optionally substituted heteroaryl,
or any pharmaceutically acceptable salt or solvate thereof.

2-16. (canceled)

17. The method according to claim 1, wherein said optionally substituted aryl is selected from a 6-, or 10-membered aryl.

18. The method according to claim 1, wherein said optionally substituted heteroaryl is selected from a 5-, 6-, 9- or 10-membered heteroaryl, wherein the number of heteroatoms is 1-3, and wherein said heteroatoms are independently selected from the group consisting of N, S, and O.

19. The method according to claim 1, wherein said aryl and heteroaryl are be substituted with one or more substituents, which may be the same or different, and are independently selected from the group consisting of C₁-C₁₀ alkyl, C₂-C₁₀ alkenyl, C₂-C₁₀ alkynyl, phenyl, amino (—NH₂), azido (—N₃), azo C₁-C₁₀ alkyl (—N₂-alkyl), cyanato (—OCN), isocyanato (—NCO), nitroxy (—ONO₂), —CH₂NH(C₁-C₁₀ alkyl), —CH₂N(C₁-C₁₀ alkyl)₂, aminoalkyl (—NH(C₁-C₁₀ alkyl), —N(C₁-C₁₀ alkyl)₂, (—N⁺(C₁-C₁₀ alkyl)₃), 1,3- or 1,4-dioxyl, morpholyl, cyano (—CN), isocyano (—NC), nitroso (—NO), CONH₂, CONH(C₁-C₁₀ alkyl), CON(C₁-C₁₀ alkyl)₂, hydroxyl (—OH), hydroperoxy (—OOH), C₁-C₁₀ peroxy alkyl (—OO-alkyl), C₁-C₁₀ alkyl hydroxyl (-alkyl-OH), C₁-C₁₀ alkoxy (—O-alkyl), carboxylic acid (—COOH), C₁-C₁₀ alkyl esters (—COO-alkyl), oxetanyl, C₁-C₁₀ alkyl acyl (—CO-alkyl), carbamoyloxy (—OC(O)NH₂), —OC(O)NH(C₁-C₁₀ alkyl), —OC(O)N(C₁-C₁₀ alkyl)₂, sulfanyl (—SH), C₁-C₁₀ alkyl thioethers (—S-alkyl), C₁-C₁₀ alkyl thioesters (—C(O)S-alkyl), sulfinic acid (—SO₂H), thiocarboxylic acid (—C(O)SH), sulfonic acid (—SO₃H), C₁-C₁₀ alkyl sulfonate (—SO₃-alkyl), phosphate (—OPO(OH)₂), phosphonic acid (—PO(OH)₂), C₁-C₁₀ alkyl phosphonate (—PO(O-alkyl)₂), phosphinic acid (—P(O)(H)OH), SO₂NH₂, hydroxamic acid (—CONHOH), C₁-C₁₀ alkyl sulfonylureas (—NHCONHSO₂(alkyl)), C₁-C₁₀ acylsulfonamides (—SO₂—NHCO-(alkyl), hydroxyl amine (—NHOH), nitro (—NO₂), imino (—N═CH₂), methyl halide having 1-3 halogen atoms, and halogens; wherein two of said C₁-C₁₀ alkyl and/or said C₁-C₁₀ alkoxy may be linked with a bridge member Z when adjacent, wherein Z is —(CH₂)ₙ—, and n is an integer from 1-6.

20. The method according to claim 1, wherein L¹, L², and L⁴ are substituted with one or more substituents, which may be the same or different, and are independently selected from the group consisting of C₁-C₁₀ alkyl, C₂-C₁₀ alkenyl, C₂-C₁₀ alkynyl, phenyl, amino (—NH₂), azido (—N₃), azo C₁-C₁₀ alkyl (—N₂-alkyl), cyanato (—OCN), isocyanato (—NCO), nitroxy (—ONO₂), —CH₂NH(C₁-C₁₀ alkyl), —CH₂N(C₁-C₁₀ alkyl)₂, aminoalkyl (—NH(C₁-C₁₀ alkyl), —N(C₁-C₁₀ alkyl)₂, (—N⁺(C₁-C₁₀ alkyl)₃), 1,3- or 1,4-dioxyl, morpholyl, cyano (—CN), isocyano (—NC), nitroso (—NO), CONH₂, CONH(C₁-C₁₀ alkyl), CON(C₁-C₁₀ alkyl)₂, hydroxyl (—OH), hydroperoxy (—OOH), C₁-C₁₀ peroxy alkyl (—OO-alkyl), C₁-C₁₀ alkyl hydroxyl (-alkyl-OH), C₁-C₁₀ alkoxy (—O-alkyl), carboxylic acid (—COOH), C₁-C₁₀ alkyl esters (—COO-alkyl), oxetanyl, C₁-C₁₀ alkyl acyl (—CO-alkyl), carbamoyloxy (—OC(O)NH₂), —OC(O)NH(C₁-C₁₀ alkyl), —OC(O)N(C₁-C₁₀ alkyl)₂, sulfanyl (—SH), C₁-C₁₀ alkyl thioethers (—S-alkyl), C₁-C₁₀ alkyl thioesters (—C(O)S-alkyl), sulfinic acid (—SO₂H), thiocarboxylic acid (—C(O)SH), sulfonic acid (—SO₃H), C₁-C₁₀ alkyl sulfonate (—SO₃-alkyl), phosphate (—OPO(OH)₂), phosphonic acid (—PO(OH)₂), C₁-C₁₀ alkyl phosphonate (—PO(O-alkyl)₂), phosphinic acid (—P(O)(H)OH), SO₂NH₂, hydroxamic acid (—CONHOH), C₁-C₁₀ alkyl sulfonylureas (—NHCONHSO₂(alkyl)), C₁-C₁₀ acylsulfonamides (—SO₂—NHCO-(alkyl), hydroxyl amine (—NHOH), nitro (—NO₂), imino (—N═CH₂), methyl halide having 1-3 halogen atoms, and halogens; wherein two of said C₁-C₁₀ alkyl and/or said C₁-C₁₀ alkoxy may be linked with a bridge member Z when adjacent, wherein Z is —(CH₂)ₙ—, and n is an integer from 1-6.

21. The method according to claim 1, wherein said optionally substituted aryl or heteroaryl are selected from the group consisting of moieties derived from benzene, naphthalene, pyrrole, furane, thiophene, thiazole, isothiazole, oxazole, isooxazole, pyrazole, imidazole, 1,2,3-oxadiazole, 1,2,4-oxadiazole, 1,2,5-oxadiazole, 1,3,4-oxadiazole, 1,2,3-triazole, 1,2,4-triazole, pyridine, pyridazine, pyrimidine, pyrazine, 1,2,4-triazine, 1,3,5-triazine, 1H-indole, indolizine, 1H-indazole, benzimidazole, 4-azaindole, 5-azaindole, 6-azaindole, 7-azaindole, 7-azaindazole, pyrazolo[1,5-a]pyrimidine, benzofuran, isobenzofuran, benzo[b]thiophene, benzo[c]thiophene, benzo[d]isoxazole, benzo[c]isoxazole, benzo[d]oxazole, benzo[c]isothiazole, benzo[d]thiazole, benzo[c][1,2,5]thiaciazole, 1H-benzotriazole, quinolone, isoquinoline, quinoxaline, phthalazine, quinazoline, cinnoline, 1,8-naphthyridine, pyrido[3,2-d]pyrimidine, pyrido[4,3-d]pyrimidine, pyrido[3,4-b]pyrazine, and pyrido[2,3-b]pyrazine.

22. The method according to claim 1, wherein L, L², and L⁴ are independently selected from the group consisting of a bond, C₁-C₈ alkylene, optionally comprising one or more moieties selected from the group consisting of an amide, a thioamide, an amine, an urea, a carbamate, an aldimine and 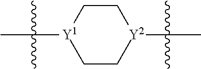
wherein Y¹ and Y² are independently selected from CH and N; or combinations thereof.

23. The method according to claim 22, wherein L¹, L², and L⁴ are independently selected from the group consisting of a bond, C₁-C₈ alkylene, a moiety of formula (A): 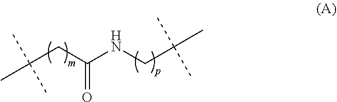
wherein m and p are an integer independently selected from 0-8, with the proviso that m+p is 8 or less, or formula (B): 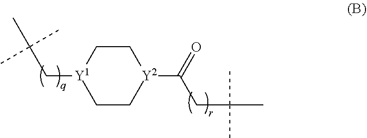
wherein q and r are an integer independently selected from 0-8, with the proviso that q+r is 8 or less, and wherein Y¹ and Y² are independently selected from CH and N.

24. A compound of formula (II): 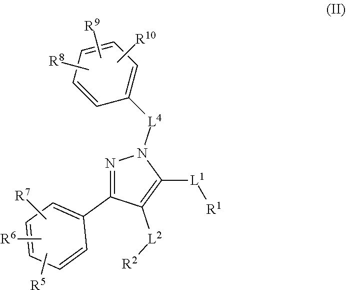
wherein L¹ is a bond,
L² is a bond or a compound of formula (A): 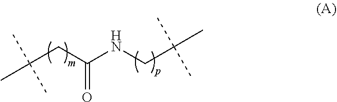
wherein m and p are an integer independently selected from 1, 2, 3, and 4,
L⁴ is selected from the group consisting of a bond, optionally substituted C₁-C₈ alkylene, optionally substituted C₂-C₈ alkenylene, optionally substituted C₂-C₈ alkynylene, optionally comprising one or more moieties selected from the group consisting of an amide, a thioamide, an ester, an amine, an urea, a carbamate, an aldimine, a ketone and 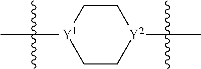
wherein Y¹ and Y² are independently selected from CH and N; or combinations thereof,
with the proviso that if L⁴ is a bond, then L² is not a bond,
R¹ and R² are independently selected from the group consisting of H, optionally substituted aryl and optionally substituted heteroaryl,
R⁵, R⁶, R⁷, R⁸, R⁹ and R¹⁰ may be the same or different and are independently selected from the group consisting of H, C₁-C₁₀ alkyl, C₂-C₁₀ alkenyl, C₂-C₁₀ alkynyl, phenyl, amino (—NH₂), —CH₂NH(C₁-C₁₀ alkyl), —CH₂N(C₁-C₁₀ alkyl)₂, aminoalkyl (—NH(C₁-C₁₀ alkyl) or —N(C₁-C₁₀ alkyl)₂, cyano (—CN), CONH₂, CONH(C₁-C₁₀ alkyl), CON(C₁-C₁₀ alkyl)₂, hydroxyl (—OH), C₁-C₁₀ alkyl hydroxyl (-alkyl-OH), C₁-C₁₀ alkoxy(—O-alkyl), carboxylic acid (—COOH), C₁-C₁₀ alkyl esters (—COO-alkyl), C₁-C₁₀ alkyl acyl (—CO-alkyl), C₁-C₁₀ thioethers (—S-alkyl), sulfonic acid (—SO₃H), C₁-C₁₀ alkyl sulfonate (—SO₃-alkyl), phosphonic acid (—PO(OH)₂), C₁-C₁₀ alkyl phosphonate (—PO(O-alkyl)₂), phosphinic acid (—P(O)(H)OH), SO₂NH₂, hydroxamic acid (—CONHOH), C₁-C₁₀ alkyl sulfonylureas (—NHCONHSO₂(alkyl)), C₁-C₁₀ acylsulfonamides (—SO₂—NHCO-(alkyl), hydroxyl amine (—NHOH), nitro (—NO₂), and halogens; wherein two of said C₁-C₁₀ alkyl and/or said C₁-C₁₀ alkoxy may be linked with a bridge member Z when adjacent, wherein Z is —(CH₂)ₙ—, and n is an integer from 1-6,
or any pharmaceutically acceptable salt or solvate thereof.

25. The compound according to claim 24, wherein L⁴ is a bond or a compound of formula (B): 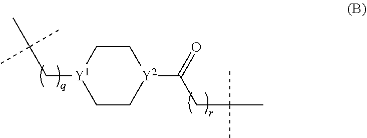
wherein q and r are an integer independently selected from 0, 1, 2, 3, and 4,
and wherein Y¹ is CH and Y² is N.

26. The compound according to any one of claim 24, wherein said aryl and heteroaryl are be substituted with one or more substituents, which may be the same or different, and are independently selected from the group consisting of C₁-C₁₀ alkyl, C₂-C₁₀ alkenyl, C₂-C₁₀ alkynyl, phenyl, amino (—NH₂), azido (—N₃), azo C₁-C₁₀ alkyl (—N₂-alkyl), cyanato (—OCN), isocyanato (—NCO), nitroxy (—ONO₂), —CH₂NH(C₁-C₁₀ alkyl), —CH₂N(C₁-C₁₀ alkyl)₂, aminoalkyl (—NH(C₁-C₁₀ alkyl), —N(C₁-C₁₀ alkyl)₂, (—N⁺(C₁-C₁₀ alkyl)₃), 1,3- or 1,4-dioxyl, morpholyl, cyano (—CN), isocyano (—NC), nitroso (—NO), CONH₂, CONH(C₁-C₁₀ alkyl), CON(C₁-C₁₀ alkyl)₂, hydroxyl (—OH), hydroperoxy (—OOH), C₁-C₁₀ peroxy alkyl (—OO-alkyl), C₁-C₁₀ alkyl hydroxyl (-alkyl-OH), C₁-C₁₀ alkoxy (—O-alkyl), carboxylic acid (—COOH), C₁-C₁₀ alkyl esters (—COO-alkyl), oxetanyl, C₁-C₁₀ alkyl acyl (—CO-alkyl), carbamoyloxy (—OC(O)NH₂), —OC(O)NH(C₁-C₁₀ alkyl), —OC(O)N(C₁-C₁₀ alkyl)₂, sulfanyl (—SH), C₁-C₁₀ alkyl thioethers (—S-alkyl), C₁-C₁₀ alkyl thioesters (—C(O)S-alkyl), sulfinic acid (—SO₂H), thiocarboxylic acid (—C(O)SH), sulfonic acid (—SO₃H), C₁-C₁₀ alkyl sulfonate (—SO₃-alkyl), phosphate (—OPO(OH)₂), phosphonic acid (—PO(OH)₂), C₁-C₁₀ alkyl phosphonate (—PO(O-alkyl)₂), phosphinic acid (—P(O)(H)OH), SO₂NH₂, hydroxamic acid (—CONHOH), C₁-C₁₀ alkyl sulfonylureas (—NHCONHSO₂(alkyl)), C₁-C₁₀ acylsulfonamides (—SO₂—NHCO-(alkyl), hydroxyl amine (—NHOH), nitro (—NO₂), imino (—N═CH₂), methyl halide having 1-3 halogen atoms, and halogens; wherein two of said C₁-C₁₀ alkyl and/or said C₁-C₁₀ alkoxy may be linked with a bridge member Z when adjacent, wherein Z is —(CH₂)ₙ—, and n is an integer from 1-6.

27. The compound according to claim 24, wherein
R⁵, R⁶, R⁷, R⁸ are H, and R⁹ and R¹⁰ are C₁-C₁₀ alkoxy(—O-alkyl); wherein said C₁-C₁₀ alkoxy may be linked with a bridge member Z when adjacent and, wherein Z is —(CH₂)ₙ—, and n is 1.

28. The compound according to claim 24 selected from the group consisting of compounds of formula (IV), and (V): 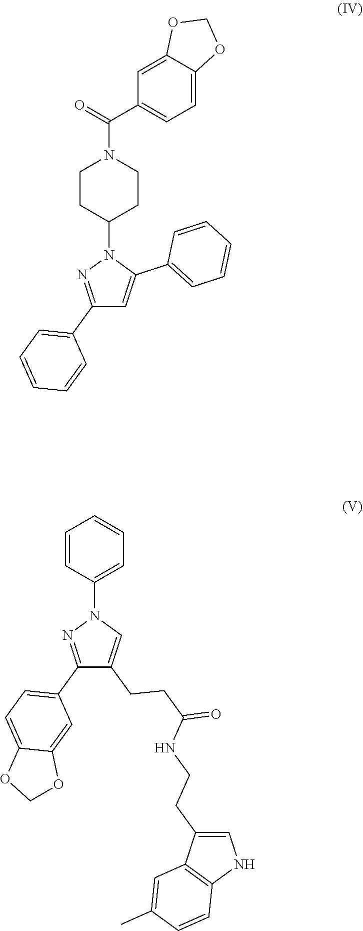 [SMILES: O=C(c1ccc2c(c1)OCO2)N1CCC(n2nc(-c3ccccc3)cc2-c2ccccc2)CC1 ; Cc1ccc2[nH]cc(CCNC(=O)CCc3cn(-c4ccccc4)nc3-c3ccc4c(c3)OCO4)c2c1]
or any pharmaceutically acceptable salts or solvates thereof.

29. The compound according to claim 24 of formula (III): 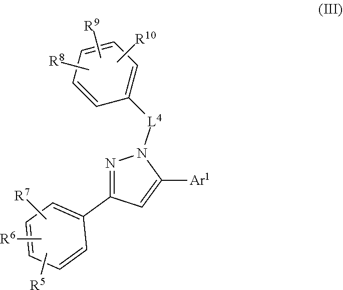
wherein
L⁴ is selected from the group consisting of C₁-C₈ alkylene, C₂-C₈ alkenylene, C₂-C₈ alkynylene, optionally comprising one or more moieties selected from the group consisting of an amide, a thioamide, an ester, an amine, a urea, a carbamate, a aldimine, a ketone and 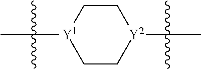
wherein Y¹ and Y² are independently selected from CH and N; or combinations thereof,
R⁵, R⁶, R⁷, R⁸, R⁹ and R¹⁰ are as defined in any one of claims 9 or 12,
Ar¹ is selected from the group consisting of optionally substituted phenyl and optionally substituted 5- or 6-membered heteroaryl,
or any pharmaceutically acceptable salt or solvate thereof.

30. The compound according to claim 29, wherein Ar¹ is optionally substituted and is selected from the group consisting of moieties derived from benzene, naphthalene, pyrrole, furane, thiophene, thiazole, isothiazole, oxazole, isooxazole, pyrazole, imidazole, 1,2,3-oxadiazole, 1,2,4-oxadiazole, 1,2,5-oxadiazole, 1,3,4-oxadiazole, 1,2,3-triazole, 1,2,4-triazole, pyridine, pyridazine, pyrimidine, pyrazine, 1,2,4-triazine, 1,3,5-triazine, and substituted benzene.

31. The compound according to claim 29, wherein said phenyl and 5- or 6-membered heteroaryl are be substituted with one or more substituents, which may be the same or different, and are independently selected from the group consisting of C₁-C₁₀ alkyl, C₂-C₁₀ alkenyl, C₂-C₁₀ alkynyl, phenyl, amino (—NH₂), azido (—N₃), azo C₁-C₁₀ alkyl (—N₂-alkyl), cyanato (—OCN), isocyanato (—NCO), nitroxy (—ONO₂), —CH₂NH(C₁-C₁₀ alkyl), —CH₂N(C₁-C₁₀ alkyl)₂, aminoalkyl (—NH(C₁-C₁₀ alkyl), —N(C₁-C₁₀ alkyl)₂, (—N⁺(C₁-C₁₀ alkyl)₃), 1,3- or 1,4-dioxyl, morpholyl, cyano (—CN), isocyano (—NC), nitroso (—NO), CONH₂, CONH(C₁-C₁₀ alkyl), CON(C₁-C₁₀ alkyl)₂, hydroxyl (—OH), hydroperoxy (—OOH), C₁-C₁₀ peroxy alkyl (—OO-alkyl), C₁-C₁₀ alkyl hydroxyl (-alkyl-OH), C₁-C₁₀ alkoxy (—O-alkyl), carboxylic acid (—COOH), C₁-C₁₀ alkyl esters (—COO-alkyl), oxetanyl, C₁-C₁₀ alkyl acyl (—CO-alkyl), carbamoyloxy (—OC(O)NH₂), —OC(O)NH(C₁-C₁₀ alkyl), —OC(O)N(C₁-C₁₀ alkyl)₂, sulfanyl (—SH), C₁-C₁₀ alkyl thioethers (—S-alkyl), C₁-C₁₀ alkyl thioesters (—C(O)S-alkyl), sulfinic acid (—SO₂H), thiocarboxylic acid (—C(O)SH), sulfonic acid (—SO₃H), C₁-C₁₀ alkyl sulfonate (—SO₃-alkyl), phosphate (—OPO(OH)₂), phosphonic acid (—PO(OH)₂), C₁-C₁₀ alkyl phosphonate (—PO(O-alkyl)₂), phosphinic acid (—P(O)(H)OH), SO₂NH₂, hydroxamic acid (—CONHOH), C₁-C₁₀ alkyl sulfonylureas (—NHCONHSO₂(alkyl)), C₁-C₁₀ acylsulfonamides (—SO₂—NHCO-(alkyl), hydroxyl amine (—NHOH), nitro (—NO₂), imino (—N═CH₂), methyl halide having 1-3 halogen atoms, and halogens; wherein two of said C₁-C₁₀ alkyl and/or said C₁-C₁₀ alkoxy may be linked with a bridge member Z when adjacent, wherein Z is —(CH₂)ₙ—, and n is an integer from 1-6.

32. The compound according to claim 29 selected from the group consisting of compound of formula (IV): 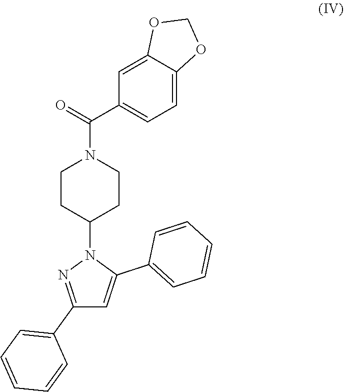 [SMILES: O=C(c1ccc2c(c1)OCO2)N1CCC(n2nc(-c3ccccc3)cc2-c2ccccc2)CC1]
or any pharmaceutically acceptable salts or solvates thereof.

33. A method of treating a cancer comprising administering to a subject having a cancer, the compound of claim 24.

34. The method of claim 34, wherein the cancer is triple negative breast cancer.
